(19) United States
(12) Patent Application Publication
(10) Pub. No.: US 2016/0120803 A1
(43) Pub. Date: May 5, 2016

(54) NON-AEROSOL FOAMS FOR TOPICAL ADMINISTRATION

(71) Applicant: CONTRACT PHARMACEUTICALS LIMITED, Mississauga (CA)

(72) Inventors: Rajiv Mathur, Mississauga (CA); Meng Zhou, Oakville (CA); Mallika Tushakiran, Mississauga (CA)

(21) Appl. No.: 14/898,987

(22) PCT Filed: Jun. 16, 2014
(86) PCT No.: PCT/CA2014/000500

§ 371 Date: Dec. 16, 2015

Related U.S. Application Data
(60) Provisional application No. 61/835,986, filed on Jun. 17, 2013.

(51) Int. Cl. A61K 9/12; A61K 31/506; A61K 31/496; A61K 31/07; A61K 31/167; A61K 31/56; A61K 31/203; A61K 9/00; A61K 31/5375

(52) U.S. Cl. CPC A61K 9/122; A61K 9/0014; A61K 31/506; A61K 31/496; A61K 31/5375; A61K 31/167; A61K 31/56; A61K 31/203; A61K 31/07

(57) ABSTRACT

Disclosed are liquid compositions suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; and (c) about 20% to about 98% (w/w) solubilizer; and, optionally, (d) about 0.5% to about 5% (w/w) emollient; wherein the composition is substantially free of propellant. Also disclosed are the foams produced by such pump actuation.

### BACKGROUND

Liquid foam is a dispersion of gas bubbles in a liquid phase. Liquid foams have been widely used in various fields, such as the engineering and petrochemical industry for froth floatation, the food industry (e.g., whipped cream, egg whites, etc.), the consumer product industry (e.g., shaving foam, shampoo, surface cleansers, disinfectants, car wax, etc.) and the pharmaceutical industry (e.g., for formulating active ingredients intended for topical delivery, e.g., in the treatment of alopecia, dermatitis, etc. and in first-aid). These industries have typically used pressurized cans to deliver products as foams. Pressurized cans and propellant gases are not environmentally friendly, and the processing of such products requires capital-intensive filling equipment and specialized dedicated manufacturing areas.

### SUMMARY

The present invention encompasses the insight that new foam compositions, and systems for producing them, would be desirable in a variety of contexts. Among other things, the present invention recognizes the value and need for foam compositions prepared or provided from non-aerosol containers. The present invention specifically appreciates that the consumer products industry has marketed cleansing hand soaps and sanitizers as foam products in non-aerosol dispensers. However, the present invention recognizes the source of a problem with such products, as dispensed material is billowing foam that lacks the richness and tightness of aerosol foam and dissipates quickly on the palms. In billowing foams, the bubbles are large and are separated from each other by very thin aqueous membranes with little or no mechanical strength.

There remains a need for products that can be delivered as foams that do not suffer from one or more of the drawbacks identified or discussed herein. There is a particular need for the development of foam products for delivery or application of active agents (e.g., agents that are therapeutic, cosmetic, and/or diagnostic, etc).

In some aspects, the present invention provides a liquid composition suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; and (c) about 20% to about 98% (w/w) solubilizer; wherein the composition is substantially free of propellant.

In some aspects, the present invention provides a liquid composition suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; and (c) about 20% to about 98% (w/w) solubilizer; wherein the composition is substantially free of propellant, and characterized in that the density of the foam produced is about 0.076 to about 0.102 g/mL.

In some aspects, the present invention provides a liquid composition suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; (c) about 20% to about 98% (w/w) solubilizer; wherein the composition is substantially free of propellant, and characterized in that the time for the foam produced to dissipate into a liquid is about 45 minutes or longer for a volume of about 40 mL of foam.

In some aspects, the present invention provides a liquid composition suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; (c) about 20% to about 98% (w/w) solubilizer; wherein the composition is substantially free of propellant, and characterized in that the specific gravity of the composition is about 0.85 to about 1.20 g/mL.

In some aspects, the present invention provides a non-aerosol pump for delivering a foam, and contained within the pump a liquid composition suitable for topical administration and for use with a non-aerosol pump, wherein actuation of the pump produces a foam, wherein the composition is a composition described herein.

In some aspects, the present invention provides a foam composition having a density of about 0.076 to about 0.102 g/mL, which foam composition is produced by a method comprising passing a liquid composition through a non-aerosol pump, wherein the liquid composition is a composition described herein.

In some aspects, the present invention provides a foam composition characterized in that the time for the foam produced to dissipate into a liquid is about 45 minutes or longer for a volume of about 40 mL of foam, which foam composition is produced by a method comprising passing a liquid composition through a non-aerosol pump, wherein the liquid composition is a composition described herein.

In some aspects, the present invention provides a method of applying to the dermis a foam composition having a density of about 0.076 to about 0.102 g/mL, which foam composition is produced by a method comprising passing a liquid composition through a non-aerosol pump, wherein the liquid composition is a composition described herein.

In some aspects, the present invention provides a method of applying to the dermis a foam composition characterized in that the time for the foam produced to dissipate into a liquid is about 45 minutes or longer for a volume of about 40 mL of foam, which foam composition is produced by a method comprising passing a liquid composition through a non-aerosol pump, wherein the liquid composition is a composition described herein.

### DETAILED DESCRIPTION

### Definitions and Usage

The term “comparable”, as used herein, refers agents, entities, situations, sets of conditions, etc that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc to be considered comparable. In general, when relative language is used herein (e.g., an agent or circumstance is said to result in “higher”, “lower”, “better”, “worse”, “reduced”, “enhanced”, “increased”, “decreased”, etc effects or values), those skilled in the art will understand that such comparison is made under comparable (e.g., identical but for the references agent or circumstance) conditions.

As used herein, the term “effective amount” means an amount that is sufficient, when administered to a population suffering from or susceptible to a disease, disorder, and/or condition in accordance with a dosing regimen, to treat the disease, disorder, and/or condition. In some embodiments, an effective amount is one that reduces the incidence and/or severity of, and/or delays onset of, one or more symptoms of the disease, disorder, and/or condition. Those of ordinary skill in the art will appreciate that the term “effective amount” does not require successful treatment be achieved in a particular individual. Rather, an effective amount may be that amount that provides a particular desired pharmacological response in a significant number of subjects when administered to patients in need of such treatment. It is specifically understood that particular subjects may, in fact, be “refractory” to an “effective amount.” To give but one example, a refractory subject may have a low bioavailability such that clinical efficacy is not obtainable. In some embodiments, reference to an effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, an effective amount may be formulated and/or administered in a single dose. In some embodiments, an effective amount may be formulated and/or administered in a plurality of doses, for example, as part of a dosing regimen.

The term “foam booster” refers to a substance that increases foam production or stabilizes lather. Such a substance may also be called a “foam stabilizer”.

The term “foaming agent” refers to (1) a substance that facilitates the formation of foam or helps maintain the integrity of the foam (e.g., by strengthening individual foam bubbles) or (2) a surfactant that (typically in small amounts) reduces the surface tension of a liquid or increases its stability (e.g., by inhibiting coalescence of bubbles).

The term “emollient” refers to a (typically hydrophobic) substance that softens or soothes the skin.

A “regimen”, as that term is used herein, refers to a dosing regimen (e.g., a therapeutic and/or prophylactic dosing regimen” whose administration across a relevant population is correlated with a desired or beneficial therapeutic outcome.

The term “solubilizer” refers to a substance that improves solubility of another substance or aids dispersion of an insoluble material into a medium or vehicle.

Unless otherwise specified, the word “or” is intended to be inclusive. For example, “A or B” means A or B or both A and B.

Unless otherwise specified, the word “includes” (or any variation thereon, e.g., “include”, “including”, etc.) is intended to be open-ended. For example, “A includes 1, 2 and 3” means that A includes but is not limited to 1, 2 and 3.

Unless otherwise specified, the word “about”, when used to modify a numeric quantity, means±5%.

### Compositions—Generally

In some aspects, the present invention provides a liquid composition suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; and (c) about 20% to about 98% (w/w) solubilizer; wherein the composition is substantially free of propellant.

In some aspects, the present invention provides a liquid composition suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; and (c) about 20% to about 98% (w/w) solubilizer; wherein the composition is substantially free of propellant, and characterized in that the density of the foam produced is about 0.076 to about 0.102 g/mL.

In some aspects, the present invention provides a liquid composition suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; (c) about 20% to about 98% (w/w) solubilizer; wherein the composition is substantially free of propellant, and characterized in that the time for the foam produced to dissipate into a liquid is about 45 minutes or longer for a volume of about 40 mL of foam.

In some aspects, the present invention provides a liquid composition suitable for topical administration and for use with a non-aerosol pump, wherein actuation of the pump produces a foam, the composition comprising: (a) about 1% to about 8% (w/w) foam booster; (b) about 0.4% to about 5% (w/w) foaming agent; (c) about 20% to about 98% (w/w) solubilizer; wherein the composition is substantially free of propellant, and characterized in that the specific gravity of the composition is about 0.85 to about 1.20 g/mL.

In some embodiments, the composition comprises: (a) about 3% to about 6% (w/w) foam booster; (b) about 1.7% to about 5% (w/w) foaming agent; (c) about 5% to about 30% (w/w) solubilizer other than water; and balance water.

In some embodiments, the composition comprises: (a) about 1% to about 2% (w/w) foam booster; (b) about 0.4% to about 1.9% (w/w) foaming agent; (c) about 5% to about 30% (w/w) solubilizer other than water; and balance water.

In some embodiments, the composition comprises: (a) about 3% to about 7% (w/w) foam booster; (b) about 2% to about 4.5% (w/w) foaming agent; (c) about 5% to about 30% (w/w) solubilizer other than water; and balance water.

In some embodiments, the composition comprises: (a) about 1.5% to about 6% (w/w) foam booster; (b) about 1.5% to about 5.0% (w/w) foaming agent; (c) about 10% to about 20% (w/w) solubilizer other than water; and balance water.

In some embodiments, the composition comprises: (a) about 2.0% to about 7.5% (w/w) foam booster; (b) about 2.0% to about 5.0% (w/w) foaming agent; (c) about 25% to about 45% (w/w) solubilizer other than water; and balance water.

In some embodiments, the composition comprises: (a) about 1.0% to about 2.0% (w/w) foam booster; (b) about 0.5% to about 2.0% (w/w) foaming agent; (c) about 10% to about 20% (w/w) solubilizer other than water; and balance water.

### Components of Compositions—Generally

In some embodiments, the foam booster is selected from Cocoamidopropyl Betaine, Cocamidopropylamine Oxide, Sodium Lauroyl Sarcosinate, Cetearyl Alcohol, Lauramine Oxide, Laureth-2, Laureth-4, Microcrystalline Cellulose, PEG-150 Distearate, PVP, Coco dimethyl carboxymethyl betaine, Cocoamidopropyl Hydroxysultaine, Cocamide Monoethanolamide, Cocamidoethyl Betaine, Lauryl Amidopropyl Betaine, Oleyl betaine, Lauryl Dimethyl Carboxymethyl Betaine, Cocamide Ethyl Betaine, Cocamide oxide, Lauryl Betaine, Lauryl Hydroxy Sultaine, Lauryl Sultaine, Ammonium Myreth Sulfate, Avocadamide DEA, Avocadoamide DIPA, Caprylyl Pyrrolidone, Cocamide DEA, Cocamide DIPA, Cocamide MEA, Dilinoleyl Amidopropyl Trimonium Chloride, Gynostemma Pentaphyllum Leaf Extract, Hydrogenated Coconut Acid, Hydrolyzed Keratin, Lauramide DEA, Lauramide MEA, Lauryl Myristyl Amine Oxide, Lauryl Pyrrolidone, Lauryldimonium Hydroxypropyl Cocoglucosides Chloride, Linoleamide DEA, Myristamine Oxide, PEG-2 Cocamide, PEG-3 Glyceryl Cocoate, PEG-30 Glyceryl Soyate, PEG-7 Glyceryl Soyate, PEG-80 Glyceryl Cocoate, Polyquaternium-43, Potassium Soyate, PPG-2 Hydroxyethyl Coco Isostearamide, Sesamide DIPA, Sodium C12-15 Pareth Sulfate, Sodium C14-16 Olefin Sulfonate, Sodium Cocoamphoacetate, Sodium Methylpalmitoyl Taurate, Sodium Methylstearoyl Taurate, Sodium Stearoyl Lactylate, Sodium Tridecyl Sulfate, Sodium Olivamphoacetate, and combinations thereof.

In some embodiments, the foam booster is suitable for pharmaceutical applications; for example, in some embodiments, the foam booster is selected from Cocoamidopropyl Betaine, Cocamidopropylamine Oxide, Sodium Lauroyl Sarcosinate, Cetearyl Alcohol, Lauramine Oxide, Laureth-2, Laureth-4, Microcrystalline Cellulose, PEG-150 Distearate, PVP, and combinations thereof.

In some embodiments, the foaming agent is selected from one or more Polyoxyethylene Sorbitan Fatty Acid Esters (such as Polysorbate 20, Polysorbate 40, Polysorbate 60, Polysorbate 80, and the like), one or more Polyoxylpropylene-polyoxyethylene Block copolymers (such as Poloxamer 124, Poloxamer 188, Poloxamer 237, Poloxamer 338, Poloxamer 407, and the like), one or more PEG ether of fatty alcohols (such as Brij L9(PEG 9 Lauryl ether), Brij L23(PEG 23 Lauryl ether, and the like), Sodium lauryl Sulphate, Sodium Laureth Sulphate, Disodium Cocoamphodiacetate, Disodium Lauryl Sulfosuccinate, Myristic Acid, Sodium Cocoyl Sarcosinate, Sodium Laureth-2 Sulfate, Sodium Laureth-3 Sulfate, Sodium Lauryl Sulfoacetate, Sucrose Stearate, one or more Polyoxyethylene Hydrogenated Castor Oils (such as Polyoxyl 40 Hydrogenated castor oil, Polyoxyl 60 Hydrogenated castor oil, and the like), Polyoxyl Lauryl Ether, PEG Ether of Lauryl Alcohol, Ammonium Laureth-2 Sulfate, Ammonium Lauryl Sulfosuccinate, Capramide DEA, Cocamidopropyl Dimethylaminohydroxypropyl Hydrolyzed Collagen, Cocoyl Hydrolyzed Soy Protein, Dihydroxyethyl Soyamine Dioleate, Dilinoleamidopropyl Dibetaine, Disodium Cocoyl Glutamate, Disodium PEG-5 Laurylcitrate Sulfosuccinate, Hydroxypropyl Guar, Laureth-1 Phosphate, Lauryl Betaine, Lauryl Phosphate, Oleoyl Sarcosine, PEG-7 Glyceryl Cocoate, Potassium Cocoyl Glycinate, Potassium Cocoyl Hydrolyzed Collagen, Potassium Lauroyl Sarcosinate, Potassium Lauryl Sulfate, Ricinoleamidopropyl Betaine, Sodium C12-13 Pareth-8 Carboxylate, Sodium Cetyl Sulfate, Sodium Cocamphoacetate, Sodium Cocomonoglyceride Sulfate, Sodium Coco-sulfate, Sodium Hydroxypropylphosphate Laurylglucoside Crosspolymer, Sodium Hydroxypropylsulfonate Cocoglucoside Crosspolymer, Sodium Hydroxypropylsulfonate Decylglucoside Crosspolymer, Sodium Lauramphoacetate, Sodium Lauroyl Glutamate, Sodium Lauroyl Hydrolyzed Silk, Sodium Lauroyl Lactylate, Sodium Lauroyl Methyl Isethionate, Sodium Lauroyl Methylaminopropionate, Sodium Lauryl Phosphate, Sodium Laurylglucoside Hydroxypropyl Sulfonate, Sodium Methyl Lauroyl Taurate, Sodium Methylcocoyl Taurate, Sodium Methylmyristoyl Taurate, Sodium Myristate, Sodium Myristoyl Glutamate, Sodium Myristoyl Sarcosinate, Sodium Myristyl Sulfate, Sodium Palmitoyl Sarcosinate, Sodium PEG-7 Olive Oil Carboxylate, Sodium Stearoyl Glutamate, Sodium Trideceth Sulfate, Sodium Trideceth-4 Carboxylate, Sodium Trideceth-7 Carboxylate, Sucrose Cocoate, TEA-Dodecylbenzenesulfonate, TEA-Cocoyl Glutamate, TEA-Cocoyl Sarcosinate, TEA-Laureth Sulfate, Trideceth-4 Carboxylic Acid, Trideceth-8 Carboxylic Acid, Yogurt Powder, Yucca Schidigera Extract, and combinations thereof.

In some embodiments, the foaming agent is suitable for pharmaceutical applications; for example, in some embodiments, the foaming agent is selected from one or more Polyoxyethylene Sorbitan Fatty Acid Esters (such as Polysorbate 20, Polysorbate 40, Polysorbate 60, Polysorbate 80, and the like), one or more Polyoxylpropylene-polyoxyethylene Block copolymers (such as Poloxamer 124, Poloxamer 188, Poloxamer 237, Poloxamer 338, Poloxamer 407, and the like), one or more PEG ether of fatty alcohols (such as Brij L9(PEG 9 Lauryl ether), Brij L23(PEG 23 Lauryl ether), and the like), Sodium lauryl Sulphate, Sodium Laureth Sulphate, Disodium Cocoamphodiacetate, Disodium Lauryl Sulfosuccinate, Myristic Acid, Sodium Cocoyl Sarcosinate, Sodium Laureth-2 Sulfate, Sodium Laureth-3 Sulfate, Sodium Lauryl Sulfoacetate, Sucrose Stearate, one or more Polyoxyethylene Hydrogenated Castor Oils (such as Polyoxyl 40 Hydrogenated castor oil, Polyoxyl 60 Hydrogenated castor oil, and the like), and combinations thereof.

In some embodiments, the solubilizer is selected from Water, Ethanol, Diethylene Glycol monomethyl ether, Glycerine, Propylene Glycol, Isopropyl alcohol, Polyethylene Glycols, Benzyl alcohol, Dimethyl Sulfoxide, PEG 40 Castor oil, PEG 40 Hydrogenated Castor Oil, PEG 60 Hydrogenated Castor Oil, Sorbitan Stearate, Oleyl Alcohol, Dimethylformamide, and combinations thereof

In some embodiments, the solubilizer is suitable for pharmaceutical applications; for example, in some embodiments, the solubilizer is selected from Water, Ethanol, Diethylene Glycol monomethyl ether, Glycerine, Propylene Glycol, Isopropyl alcohol, Polyethylene Glycols, Benzyl alcohol, Dimethyl Sulfoxide, PEG 40 Castor oil, PEG 40 Hydrogenated Castor Oil, PEG 60 Hydrogenated Castor Oil, Sorbitan Stearate, Oleyl Alcohol, and combinations thereof.

Compositions—with Emollient

In some embodiments, the composition further comprises (d) about 0.5% to about 5% (w/w) emollient.

In some embodiments, the composition comprises: (a) about 3% to about 6% (w/w) foam booster; (b) about 1.7% to about 5% (w/w) foaming agent; (c) about 5% to about 30% (w/w) solubilizer other than water; (d) about 1% to about 2% (w/w) emollient; and balance water.

In some embodiments, the composition comprises: (a) about 1% to about 2% (w/w) foam booster; (b) about 0.4% to about 1.9% (w/w) foaming agent; (c) about 5% to about 30% (w/w) solubilizer other than water; (d) about 1% to about 2% (w/w) emollient; and balance water.

In some embodiments, the composition comprises: (a) about 3% to about 7% (w/w) foam booster; (b) about 2% to about 4.5% (w/w) foaming agent; (c) about 5% to about 30% (w/w) solubilizer other than water; (d) about 0.5% to about 1% (w/w) emollient; and balance water.

In some embodiments, the composition comprises: (a) about 1.5% to about 6% (w/w) foam booster; (b) about 1.5% to about 5.0% (w/w) foaming agent; (c) about 20% to about 35% (w/w) solubilizer other than water; (d) about 1.0% to about 2.0% (w/w) emollient; and balance water.

In some embodiments, the composition comprises: (a) about 2.0% to about 7.5% (w/w) foam booster; (b) about 2.0% to about 5.0% (w/w) foaming agent; (c) about 25% to about 45% (w/w) solubilizer other than water; (d) about 0.5% to about 1.0% (w/w) emollient; and balance water.

In some embodiments, the composition comprises: (a) about 1.0% to about 2.0% (w/w) foam booster; (b) about 0.5% to about 2.0% (w/w) foaming agent; (c) about 20% to about 45% (w/w) solubilizer other than water; (d) about 1.0% to about 5.0% (w/w) emollient; and balance water.

In some embodiments, the emollient is selected from Dimethicone, Cyclomethicone, Arachis Hypogaea (Peanut) Oil, Canola Oil, Carthamus Tinctorius (Safflower) Seed Oil, Cocos Nucifera (Coconut) Oil, Citrus Aurantium Amara (Bitter Orange) Peel Oil, Citrus Medica Limonum (Lemon) Peel Oil, Citrus Aurantifolia (Lime) Oil, Elaeis Guineensis (Palm) Oil, Glycine Soja (Soybean) Oil, Mentha Piperita (Peppermint) Oil, Olea Europaea (Olive) Oil, PEG-60 Hydrogenated Castor Oil, Papaver Somniferum (Poppy) Seed Oil, Ricinus Communis (Castor) Seed Oil, Sesamum Indicum (Sesame) Seed Oil, Zea Mays (Corn) Oil, Butyrospermum Parkii (Shea) Butter, Theobroma Cacao (Cocoa) Seed Butter, PEG-75 Lanolin, PEG-2 Stearate, PEG-8 Laurate, PPG-15 Stearyl Ether, PPG-20 MethylGlucose Ether Distearate, Lanolin, Lanolin Alcohol, Acetylated Lanolin Alcohol, Lecithin, Caprylic/Capric Triglyceride, Caprylic/Capric/Stearic Triglyceride, Hydrogenated Palm Glycerides, Glycol Stearate, Butylene Glycol, Hexylene Glycol, Propylene Glycol, Cetearyl Alcohol, Cetyl Alcohol, Myristyl Alcohol, Oleyl Alcohol, Stearyl Alcohol, Glyceryl Caprylate, Glyceryl Laurate, Glyceryl Isostearate, Glyceryl Oleate, Glyceryl Stearate, Glyceryl Distearate, Polyglyceryl-3 Oleate, Polyglyceryl-10 Oleate, Isostearic Acid, Lactic Acid, Sodium Lactate, Copernicia Cerifera (Carnauba) Wax, Cera Alba (Beeswax), Euphorbia Cerifera (Candelilla) Wax, Polyquaternium-7, Choleth-24, Methyl Gluceth-10, Methyl Gluceth-20, Diisopropyl Adipate, Methyl Laurate, Cetearyl Ethylhexanoate, Diethyl Sebacate, Ethylhexyl Hydroxystearate, Xanthan Gum, Isopropyl Myristate, Isopropyl Palmitate, Cetyl Palmitate, Octyldodecanol, Acacia, Butyl Stearate, Cholesterol, Cysteine HCl, Lauryl Lactate, Myristyl Lactate, Paraffinum Liquidum (Mineral) Oil, Petrolatum, Polybutene, PVP, Sorbitan Isostearate, Squalane, Tocopherol, Tricaprylin, Urea, Zea Mays (Corn) Starch, Hyaluronic Acid, Sodium Hyaluronate, PEG 90 Diisostearte, PEG/PPG-8/3 Diisostearate, C₁₂₋₁₅ Alkyl Benzoate, Cetyl Ricinoleate Benzoate, Ethylhexyl Hydroxystearate Benzoate, Methyl Gluceth-20 Benzoate, Dipropylene Glycol Dibenzoate, Dimethicone PEG/PPG-20/23 Benzoate, Dimethicone PEG-8 Benzoate, Dipropylene Glycol Dibenzoate, PPG-15 Stearyl Ether Benzoate, Bis-PEG/PPG-20/20 dimethicone, Stearoxy Dimethicone, Behenoxy Dimethicone, Stearyl Dimethicone, Cetyl Dimethicone, Cerotyl Dimethicone, PEG-8 Dimethicone, PEG-9 Dimethicone, PEG-14 Dimethicone, PEG-12 Dimethicone/PPG-20 Crosspolymer, Undecylcrylene Dimethicone, Perfluorononyl Dimethicone, PEG-8 PG-Coco-Glucoside Dimethicone, Dimethicone/Vinyl Dimethicone Crosspolymer, Dimethicone PEG-8 Beeswax, C₂₆₋₂₈ Alkyl Dimethicone, Caprylyl Methicone, Phenyl Trimethicone, C₂₆₋₂₈ Alkyl Methicone, Amaranthus Caudatus (Amaranth) Seed Oil, Adansonia Digitata (Baobab) Seed Oil, Avena Sativa (Oat) Kernel Oil, Actinidia Chinensis (Kiwi) Seed Oil, Aleurites Moluccana (Kukui nut) Seed Oil, Argania Spinosa (Argan) Kernel Oil, Borago Officinalis (Borage) Seed Oil, Bertholetta Excelsa (Brazil Nut) Seed Oil, Brassica Oleracea Italica (Broccoli) Seed Oil, Brassica Campestris (Rapeseed) Seed Oil, Carapa Guaianensis (Andiroba) Seed Oil, Corylus Avellana (Hazel) Seed Oil, Cannabis Sativa (Hemp) Seed Oil, Camelina Sativa (Camelina) Seed Oil, Camellia Oleifera (Tea) Seed Oil, Crambe Abyssinica Seed Oil, Citrus Paradisi (Grapefruit) Seed Oil, Cucurbita Pepo (Pumpkin) Seed Oil, Cucumis Sativus (Cucumber) Seed Oil, Citrullus Lanatus (Watermelon) Seed Oil, Carica Papaya (Papaya) Seed Oil, Eucalyptus Globulus Leaf Oil, Euterpe Oleracea (Acai) Fruit Oil, Emu Oil, Fragaria Ananassa (Strawberry) Seed Oil, Hippophae Rhamnoides (Seabuckthorn) Oil, Helianthus Annuus (Sunflower) Seed Oil, Juglans Regia (Walnut) Seed Oil, Limnanthes Alba (Meadowfoam) Seed Oil, Linum Usitatissimum (Linseed) Seed Oil, Macadamia Integrifolia (Macadamia) Seed Oil, Mangifera Indica (Mango) Seed Oil, Mauritia Flexuosa Fruit Oil, Moringa Pterygosperma (Moringa) Seed Oil, Nigella Sativa (Cumin) Seed Oil, Neatsfoot Oil, Orbignya Oleifera (Babassu) Seed Oil, Oenothera Biennis (Evening Primrose) Oil, Oryza Sativa (Rice) Bran Oil, Olus Oil, Passiflora Edulis (Passion) Seed Oil, Passiflora Incarnata Seed Oil, Pistacia Vera (Pistachio) Seed Oil, Prunus Amygdalus Dulcis (Sweet Almond) Oil, Prunus Armeniaca (Apricot) Kernel Oil, Prunus Domestica (Plum) Seed Oil, Prunus Avium (Sweet Cherry) Seed Oil, Persea Gratissima (Avocado) Oil, Punica Granatum (Pomegranate) Seed Oil, Prunus Persica (Peach) Kernel Oil, Ribes Nigrum (Black Currant) Seed Oil, Rose Canina (Rose Hips) Fruit Oil, Rosa Moschata Seed Oil, Rubus Occidentalis (Black Raspberry) Seed Oil, Rubus Idaeus (Raspberry) Seed Oil, Salvia Hispanica (Chia) Seed Oil, Scierocarya Birrea (Marula) Kernel Oil, Silybum Marianum (Milk Thistle) Seed Oil, Simmondsia Chinensis (Jojoba) Seed Oil, Sisymbrium Irio (Rocket) Seed Oil, Solanum Lycopersicum (Tomato) Seed Oil, Triticum Vulgare (Wheat) Germ Oil, Vaccinium Corymbosum (Blueberry) Seed Oil, Vaccinium Macrocarpan (Cranberry) Seed Oil, Vaccinium Myrtillus (Bilberry) Seed Oil, Vitis Vinifera (Grape) Seed Oil, Algae Extract, Aloe Barbadensis Leaf Extract, Althaea Officinalis Extract, Anthemis Nobilis Flower Extract, Arnica Montana Flower Extract, Ascophyllum Nodosum Extract, Avena Sativa (Oat) Kernel Extract, Caviar Extract, Centella Asiatica Extract, Cocos Nucifera (Coconut) Extract, Coffea Arabica (Coffee) Extract, Cymbopogon Citratus Extract, Daucus Carota Sativa (Carrot) Extract, Echinacea Angustifolia Extract, Echinacea Purpurea Extract, Fucus Vesiculosus Extract, Hydrolyzed Adansonia Digitata Extract, Lactobacillus/Phoenix Dactylifera (Date) Fruit Ferment Extract, Linum Usitatissimum (Linseed) Seed Extract, Pisum Sativum (Pea) Extract, Pollen Extract, Prunus Amygdalus Dulcis (Sweet Almond) Fruit Extract, Prunus Armeniaca (Apricot) Fruit Extract, Pyrus Malus (Apple) Fruit Extract, Sargassum Vulgare Extract, Sea Cucumber Extract, Shorea Robusta Leaf Extract, Stevia Rebaudiana Extract, Symphytum Officinale Leaf Extract, Urtica Dioica (Nettle) Extract, Vaccinium Angustifolium (Blueberry) Fruit Extract, Vaccinium Macrocarpan (Cranberry) Fruit Extract, Vaccinium Myrtillus Extract, Astrocaryum Murumuru Seed Butter, Astrocaryum Tucuma Seed Butter, Elaeis Guineensis (Palm) Butter, Garcini Indica Seed Butter, Irvingia Gabonensis Kernel Butter, Mangifera Indica (Mango) Seed Butter, Shorea Stenoptera Butter, Shorea Stenoptera Seed Butter, Theobroma Grandiflorum Seed Butter, Almond Oil PEG-8 Esters, one or more Babassu Oil Glycereth-8 Esters, one or more Coconut Oil Glycereth-8 Esters, one or more Cottonseed Oil Glycereth-8 Esters, one or more Grape Seed Oil PEG-8 Esters, one or more Cannabis Sativa Seed Oil PEG-8 Esters, one or more Macadamia Seed Oil Glycereth-8 Esters, one or more Peach Kernel Oil Glycereth-8 Esters, one or more Avocado Oil Glycereth-8 Esters, one or more Sesame Oil Glycereth-8 Esters, one or more Soybean Oil Glycereth-8 Esters, one or more Sunflower Seed Oil Glycereth-8 Esters, one or more Olive Oil PEG-7 Esters, one or more PEG-6 Capric Glycerides, one or more PEG-6 Caprylic/Capric Glycerides, one or more PEG-6 Caprylic/Capric Triglycerides, PEG-3 Glyceryl Cocoate, PEG-7 Glyceryl Cocoate, PEG-30 Glyceryl Cocoate, PEG-80 Glyceryl Cocoate, PEG-10 Lanolin, PEG-20 Lanolin, PEG-30 Lanolin, PEG-60 Lanolin, PEG-8 Distearate, PEG-90 Diisostearate, PEG/PPG-8/3 Diisostearate, PEG-8 Dilaurate, PEG-12 Laurate, PEG-12 Dilaurate, PEG/PPG-8/3 Laurate, PEG-5 Isononanoate, PEG-2 Diisononanoate, PEG-2 Dioctanoate, PEG-40 Butyloctanol, one or more PEG-10 Sunflower Glycerides, one or more PEG-16 Macadamia Glycerides, PEG-8 Dioleate, one or more PEG-75 Shea Butter Glycerides, one or more PEG-70 Mango Glycerides, PEG-5 Soy Sterol, PEG-25 PABA, PEG-35 Castor Oil, PPG-2 Myristyl Ether Propionate, PPG-3 Myristyl Ether, PPG-5-Ceteth-20, PPG-5 Pentaerythrityl Ether, PPG-10 Cetyl Ether, PPG-10 MethylGlucose Ether, PPG-11 Stearyl Ether, PPG-14 Butyl Ether, PPG-20 MethylGlucose Ether, Lanolin Oil, Lanolin Wax, Acetylated Lanolin, Hydroxylated Lecithin, Capric/Capric Triglyceride, Caprylic/Capric/Lauric Triglyceride, Caprylic/Capric/Linoleic Triglyceride, one or more Hydrogenated Coco-Glycerides, one or more Olive glycerides, one or more Soybean Glycerides, one or more Wheat Germ Glycerides, Glycol Distearate, Glycol Palmitate, Caprylyl Glycol, Butylene Glycol Cocoate, Butylene Glycol Dicaprylate/Dicaprate, Propylene Glycol Heptanoate, Propylene Glycol Caprylate, Propylene Glycol Dicaprate, Propylene Glycol Dicaprylate/Dicaprate, Propylene Glycol Diethylhexanoate, Propylene Glycol Stearate, Neopentyl Glycol Diheptanoate, Neopentyl Glycol Dicaprate/Dicaprylate, Neopentyl Glycol Dioctanoate, Batyl Alcohol, Behenyl Alcohol, Chimyl Alcohol, Lauryl Alcohol, Glyceryl Caprylate/Caprate, Glyceryl Dilaurate, Glyceryl Triacetyl Hydroxystearate, Glyceryl Undecylenate, Oleyl Glyceryl Ether, Bis-Diglyceryl Polyacyladipate-1,Bis-Diglyceryl Polyacyladipate-2, Polyglyceryl-2 Triisostearate, Polyglyceryl-3 Caprate, Polyglyceryl-3 Laurate, Polyglyceryl-3 Stearate, Polyglyceryl-3 Polyricinoleate, Polyglyceryl-4 Caprate, Polyglyceryl-6 Octastearate, Polyglyceryl-6 Dioleate, Polyglyceryl-10 Decaoleate, Polyglyceryl-10 Decaisostearate, Polyglyceryl-10 Decamacadamiate, Polyglyceryl-10 Heptaoleate, Polyglyceryl-10 Mono/Dioleate, Polyglyceryl-10 Pentastearate, Polyglyceryl-10 Tetraoleate, Diisostearoyl Polyglyceryl-3 Dimer Diinoleate, Triisostearyl Polyglycerol-3 Dimer Dilinoleate, Arachidonic Acid, Coconut Acid, Isostearyl Linoleate Abietic Acid, Linoleic Acid, Linolenic Acid, Salicylic Acid, Sodium C₁₂₋₁₅ Alkoxypropyl iminodipropionate, Sodium Lauroyl Lactylate, Sodium Stearoyl Lactylate, Sodium Usnate, Disodium Oleamido MEA-Sulfosuccinate, Sodium Acrylates Copolymer, one or more Jojoba Wax PEG-80 Esters, Citrus Medica Limonum (Lemon) Peel Wax, Narcissus Poeticus Wax, Citrus Aurantium Dulcis (Orange) Peel Wax, Castor Isostearate Beeswax Succinate, Polyquaternium-6, Polyquaternium-37, Glycereth-26, Laureth-12 Succinate, Ceteareth-20, Ceteareth-22, Ceteareth-6 Olivate, Trideceth-6, Trideceth-9, Ceteth-24, Myreth-3 Myristate, Dihydrocholeth-30, one or more Shea Butter Glycereth-8 Esters, Cetyl Acetate, Tocopheryl Acetate, Panthenyl Triacetate, Dibutyl Adipate, Diisodecyl Adipate, Dioctyl Adipate, Methyl Caprate, Methyl Cocoate, Methyl Lactate, Methyl Myristate, Methyl Oleate, Methyl Palmitate, Methyl Soyate, Polymethylsilsesquioxane, Methylglucose Dioleate, Trimethylolpropane Triisostearate, C₁₂₋₁₅ Alkyl Ethylhexanoate, Cetyl Ethylhexanoate, Diethylhexyl 2,6-naphthalate, Diethylhexyl Carbonate, Diethylhexyl Maleate, Ethyl Macadamiate, Ethyl Linoleate, Ethyl Olivate, Ethylhexyl Ethylhexanoate, Ethylhexyl Isononanoate, Ethylhexyl Pelargonate, Ethylhexyl Palmitate, Ethylhexyl Stearate, Triethylhexanoin, Sclerotium Gum, Isopropyl Linoleate, Diisopropyl Sebacate, Guar Hydroxypropyltrimonium Chloride, Hydroxypropyl Guar Hydroxypropyltrimonium Chloride, Hydroxypropyl Guar, Borageamidopropyl Phosphatidyl-PG-Dimonium Chloride, Hydroxypropyltrimonium Hydrolyzed Wheat Protein, C₃₀₋₃₈ Olefin/Isopropyl Maleate/MA Copolymer, Hydroxypropyl Oxidized Starch PG-Trimonium Chloride, Starch Hydroxypropyltrimonium Chloride, Isopropyl C₁₂₋₁₅-Pareth-9-Carboxylate, Cetyl Ricinoleate, Isocetyl Myristate, Isocetyl Palmitate, Isocetyl Stearate, Tricetyl Phosphate, Stearyl Heptanoate, Distearyl Ether, Isostearyl Neopentanoate, Isostearyl Avocadate, Isostearyl Isostearate, Isostearyl Palmitate, Diisostearyl Malate, Diisostearyl Dimer Dilinoleate, Diisostearyl Fumarate, Diisostearyl Malate, Triisostearyl Citrate, Triisostearyl Trilinoleate, Octyl Stearate, Octyl Isononanoate, Octyl Palmitate, Octyl Cocoate, Octyl Palmitate, Octyldodecyl PPG-3 Myristyl Ether Dimer Dilinoleate, Octyldodecyl Neodecanoate, Octyldodecyl Myristate, Octyl Methoxycinnamate, Octyldodecyl Ricinoleate, Dioctyl Sebacate, Dioctyldodecyl Fluoroheptyl Citrate, Trioctyldodecyl Citrate, Butyloctyl Salicylate, 1,2-Hexanediol, AMP-Isostearoyl Hydrolyzed Collagen, Arachidyl Behenate, one or more Avocado Sterols, Benzophenone-3,Benzyl Nicotinate, Bisabolol, Butyl Myristate, Castor Isostearate Succinate, Castoryl Maleate, Ceramide 3, Cetearyl Isononanoate, Cholesteryl Stearate, Cholesteryl/Octydodecyl Lauroyl Glutamate, Cocamide MEA, Cyclopentasiloxane, Cyclotetrasiloxane, Decyl Cocoate, Decyl Oleate, Dicaprylyl Carbonate, Dicaprylyl Ether, Dimethiconol, Dipentaerythrityl Hexacaprylate/Hexacaprate, Domiphen Bromide, Ectoin, Galactosyl Fructose, one or more Glycosaminoglycans, Hexyldecanol, Hexyldecyl Isostearate, Hexyldecyl Laurate, one or more Hydrogenated C₆₋₁₄ Olefin Polymers, Hydrogenated Didecene, Hydrogenated Polydecene, Hydrolyzed Collagen, Hydrolyzed Elastin, Hydrolyzed Oat Protein, Isoamyl Laurate, Isobutyl Stearate, Isodecyl Neopentanoate, Isodecyl Octanoate, Isododecane, Lignoceryl Erucate, Magnesium Ascorbyl Phosphate, Mannan, Myristyl Myristate, Octocrylene, Oleyl Betaine, Pentaerythrityl Tetracaprylate/Tetracaprate, Pentaerythrityl Tetraisostearate, Phytosterol, Phytosteryl Hydroxystearate, Phytosteryl Isostearate, Polyamide-7, Polydecene, Polydiethysiloxane, Polyisobutene, Potassium Lauroyl Hydrolyzed Soy Protein, Potassium Soyate, Propanediol, Pyridoxine Dioctenoate, Pyridoxine Dipalmitate, Retinyl Palmitate, Sea Cucumber Chondroitin Sulfate, Sericin, Shark Cartilage Chondroitin Sulfate, Stearamide AMP, Sucrose Cocoate, Sucrose Polybehenate, Sucrose Polysoyate, Tricaprin, Tridecyl Neopentanoate, Tridecyl Salicylate, Tridecyl Stearate, Tridecyl Trimellitate, Triheptanoin, Triisostearin, Trioctanoin, Tris (PPG-3 Benzyl Ether) Citrate, Triundecanoin, Ubiquinone, and combinations thereof.

In some embodiments, the emollient is suitable for pharmaceutical applications; for example, in some embodiments, the emollient is selected from Dimethicone, Cyclomethicone, Arachis Hypogaea (Peanut) Oil, Canola Oil, Carthamus Tinctorius (Safflower) Seed Oil, Cocos Nucifera (Coconut) Oil, Citrus Aurantium Amara (Bitter Orange) Peel Oil, Citrus Medica Limonum (Lemon) Peel Oil, Citrus Aurantifolia (Lime) Oil, Elaeis Guineensis (Palm) Oil, Glycine Soja (Soybean) Oil, Mentha Piperita (Peppermint) Oil, Olea Europaea (Olive) Oil, PEG-60 Hydrogenated Castor Oil, Papaver Somniferum (Poppy) Seed Oil, Ricinus Communis (Castor) Seed Oil, Sesamum Indicum (Sesame) Seed Oil, Zea Mays (Corn) Oil, Butyrospermum Parkii (Shea) Butter, Theobroma Cacao (Cocoa) Seed Butter, PEG-75 Lanolin, PEG-2 Stearate, PEG-8 Laurate, PPG-15 Stearyl Ether, PPG-20 MethylGlucose Ether Distearate, Lanolin, Lanolin Alcohol, Acetylated Lanolin Alcohol, Lecithin, Caprylic/Capric Triglyceride, Caprylic/Capric/Stearic Triglyceride, Hydrogenated Palm Glycerides, Glycol Stearate, Butylene Glycol, Hexylene Glycol, Propylene Glycol, Cetearyl Alcohol, Cetyl Alcohol, Myristyl Alcohol, Oleyl Alcohol, Stearyl Alcohol, Glyceryl Caprylate, Glyceryl Laurate, Glyceryl Isostearate, Glyceryl Oleate, Glyceryl Stearate, Glyceryl Distearate, Polyglyceryl-3 Oleate, Polyglyceryl-10 Oleate, Isostearic Acid, Lactic Acid, Sodium Lactate, Copernicia Cerifera (Carnauba) Wax, Cera Alba (Beeswax), Euphorbia Cerifera (Candelilla) Wax, Polyquaternium-7, Choleth-24, Methyl Gluceth-10, Methyl Gluceth-20, Diisopropyl Adipate, Methyl Laurate, Cetearyl Ethylhexanoate, Diethyl Sebacate, Ethylhexyl Hydroxystearate, Xanthan Gum, Isopropyl Myristate, Isopropyl Palmitate, Cetyl Palmitate, Octyldodecanol, Acacia, Butyl Stearate, Cholesterol, Cysteine HCl, Lauryl Lactate, Myristyl Lactate, Paraffinum Liquidum (Mineral) Oil, Petrolatum, Polybutene, PVP, Sorbitan Isostearate, Squalane, Tocopherol, Tricaprylin, Urea, Zea Mays (Corn) Starch, Hyaluronic Acid, Sodium Hyaluronate, and combinations thereof.

In some embodiments, a provided composition comprises: (a) about 1% to about 8% (w/w) foam booster; (b) about 0.4% to about 5% (w/w) foaming agent; (c) about 20% to about 98% (w/w) solubilizer; and (d) about 0.5% to about 5% (w/w) emollient, wherein:
the foam booster is selected from Cocoamidopropyl Betaine, Cocamidopropylamine Oxide, Sodium Lauroyl Sarcosinate, Cetearyl Alcohol, Lauramine Oxide, Laureth-2, Laureth-4, Microcrystalline Cellulose, PEG-150 Distearate, PVP, and combinations thereof;
the foaming agent is selected from one or more Polyoxyethylene Sorbitan Fatty Acid Esters (such as Polysorbate 20, Polysorbate 40, Polysorbate 60, Polysorbate 80, and the like), one or more Polyoxylpropylene-polyoxyethylene Block copolymers (such as Poloxamer 124, Poloxamer 188, Poloxamer 237, Poloxamer 338, Poloxamer 407, and the like), one or more PEG ether of fatty alcohols (such as Brij L9(PEG 9 Lauryl ether), Brij L23(PEG 23 Lauryl ether), and the like), Sodium lauryl Sulphate, Sodium Laureth Sulphate, Disodium Cocoamphodiacetate, Disodium Lauryl Sulfosuccinate, Myristic Acid, Sodium Cocoyl Sarcosinate, Sodium Laureth-2 Sulfate, Sodium Laureth-3 Sulfate, Sodium Lauryl Sulfoacetate, Sucrose Stearate, one or more Polyoxyethylene Hydrogenated Castor Oils (such as Polyoxyl 40 Hydrogenated castor oil, Polyoxyl 60 Hydrogenated castor oil, and the like), and combinations thereof;
the solubilizer is selected from Water, Ethanol, Diethylene Glycol monomethyl ether, Glycerine, Propylene Glycol, Isopropyl alcohol, Polyethylene Glycols, Benzyl alcohol, Dimethyl Sulfoxide, PEG 40 Castor oil, PEG 40 Hydrogenated Castor Oil, PEG 60 Hydrogenated Castor Oil, Sorbitan Stearate, Oleyl Alcohol, and combinations thereof; and
the emollient is selected from Dimethicone, Cyclomethicone, Arachis Hypogaea (Peanut) Oil, Canola Oil, Carthamus Tinctorius (Safflower) Seed Oil, Cocos Nucifera (Coconut) Oil, Citrus Aurantium Amara (Bitter Orange) Peel Oil, Citrus Medica Limonum (Lemon) Peel Oil, Citrus Aurantifolia (Lime) Oil, Elaeis Guineensis (Palm) Oil, Glycine Soja (Soybean) Oil, Mentha Piperita (Peppermint) Oil, Olea Europaea (Olive) Oil, PEG-60 Hydrogenated Castor Oil, Papaver Somniferum (Poppy) Seed Oil, Ricinus Communis (Castor) Seed Oil, Sesamum Indicum (Sesame) Seed Oil, Zea Mays (Corn) Oil, Butyrospermum Parkii (Shea) Butter, Theobroma Cacao (Cocoa) Seed Butter, PEG-75 Lanolin, PEG-2 Stearate, PEG-8 Laurate, PPG-15 Stearyl Ether, PPG-20 MethylGlucose Ether Distearate, Lanolin, Lanolin Alcohol, Acetylated Lanolin Alcohol, Lecithin, Caprylic/Capric Triglyceride, Caprylic/Capric/Stearic Triglyceride, Hydrogenated Palm Glycerides, Glycol Stearate, Butylene Glycol, Hexylene Glycol, Propylene Glycol, Cetearyl Alcohol, Cetyl Alcohol, Myristyl Alcohol, Oleyl Alcohol, Stearyl Alcohol, Glyceryl Caprylate, Glyceryl Laurate, Glyceryl Isostearate, Glyceryl Oleate, Glyceryl Stearate, Glyceryl Distearate, Polyglyceryl-3 Oleate, Polyglyceryl-10 Oleate, Isostearic Acid, Lactic Acid, Sodium Lactate, Copernicia Cerifera (Carnauba) Wax, Cera Alba (Beeswax), Euphorbia Cerifera (Candelilla) Wax, Polyquaternium-7, Choleth-24, Methyl Gluceth-10, Methyl Gluceth-20, Diisopropyl Adipate, Methyl Laurate, Cetearyl Ethylhexanoate, Diethyl Sebacate, Ethylhexyl Hydroxystearate, Xanthan Gum, Isopropyl Myristate, Isopropyl Palmitate, Cetyl Palmitate, Octyldodecanol, Acacia, Butyl Stearate, Cholesterol, Cysteine HCl, Lauryl Lactate, Myristyl Lactate, Paraffinum Liquidum (Mineral) Oil, Petrolatum, Polybutene, PVP, Sorbitan Isostearate, Squalane, Tocopherol, Tricaprylin, Urea, Zea Mays (Corn) Starch, Hyaluronic Acid, Sodium Hyaluronate, and combinations thereof.
   Compositions—with Active Ingredient:

In some embodiments, a provided composition further comprises: (e) an active ingredient, for example that may be suitable for use in the treatment of a dermatological disease, disorder, or condition; or is suitable for use as an anti-fungal agent, an anti-bacterial agent, or a local anesthetic. Alternatively or additionally, in some embodiments, a provided composition may include an active ingredient suitable for use in the treatment of one or more of alopecia, dermatitis, basal cell carcinoma, acne, actinic keratosis, rosacea, and psoriasis.

As used herein, “suitable for use in the treatment of” means that the active ingredient produces a therapeutic benefit in a subject having or at risk of a disease, disorder, or condition who receives the treatment. In some embodiments, therapeutic benefit includes elimination, amelioration (e.g., reduction in frequency and/or severity), and/or delay of onset of one or more symptoms caused by or otherwise associated with the disease, disorder, or condition. In some embodiments, a therapeutic benefit is achieved through administration after one or more symptoms of a disease, disorder or condition becomes manifest. In some embodiments, therapeutic benefit is achieved through administration prior to manifestation of some or all relevant symptoms (e.g., through prophylactic administration). In some embodiments, the active ingredient is approved by a relevant national health authority (e.g., the United States Food and Drug Administration, the European Medicines Agency, Health Canada, etc.) for use in treating the disease, disorder, or condition.

In some embodiments, a provided composition is used in combination therapy, in which the use of a provided composition containing an active ingredient is combined with the use of one or more other therapeutic agents for treating the same and/or other types of diseases, disorders or conditions. Combination therapy includes administration of the therapeutic agents concurrently or sequentially. Alternatively, a provided composition can contain two or more active ingredients.

It is recognized that there will be an effective amount of an active ingredient against any particular disease, disorder or condition. In some embodiments, a provided composition includes an effective amount of an active ingredient; in some embodiments, a provided composition includes an amount (and/or a concentration) sufficient so that a single actuation of a pump containing the composition produces an aliquot of foam containing an effective amount. In some embodiments, a provided composition includes an amount (and/or a concentration) that does not produce a foam containing an effective amount of an active ingredient through a single actuation of a pump; rather, in some embodiments, more than one actuation of the pump may be required to produce a foam containing an effective amount of active ingredient.

In some embodiments, the active ingredient is suitable for use in the treatment of alopecia, dermatitis, basal cell carcinoma, acne, actinic keratosis, rosacea, or psoriasis, or is suitable for use as an anti-fungal agent, an anti-bacterial agent, or a local anesthetic.

In some embodiments, the active ingredient is suitable for use in the treatment of alopecia; dermatitis, such as Allergic contact dermatitis, Seborrheic dermatitis, Irritant contact dermatitis, Atopic dermatitis, Neurodermatitis, Perioral dermatitis, Stasis dermatitis, Diaper dermatitis, Lichen simplex chronicus, Dyshidrotic dermatitis, Nummular dermatitis, Autosensitization dermatitis, Spongiotic dermatitis, Lichenoid dermatitis; basal cell carcinoma; acne, such as blackheads, whiteheads, pustules, Acne Vulgaris, Acne Rosacea, Cystic Acne, Nodular Acne, Acne Inversa, Hormonal Acne, Acne conglobata, Acne fulminans, Gram Negative folliculitis, Nodulocystic acne, Pyoderma Faciale, Acne Cosmetica, Pomade Acne, Excoriated Acne, Acne Agminata, Acne excoriee; actinic keratosis, such as Hyperkeratotic actinic keratosis, Pigmented actinic keratosis, Lichenoid actinic keratosis, Atrophic actinic keratosis; rosacea, such as Vascular Rosacea, Acne Rosacea, Pre Rosacea, Papulopustular Rosacea, Late Rosacea, Erythematotelangiectatic Rosacea, Ocular Rosacea, Phymatous Rosacea; or psoriasis, such as Psoriasis vulgaris (Plague Psoriasis), Guttate Psoriasis, Inverse Psoriasis, Pustular Psoriasis, Erythrodermic Psoriasis, Nail Poriasis, Psoriasis of the scalp, Psoriatic Arthritis, Seborrheic Psoriasis, Palmoplantar Psoriasis, skin fold Psoriasis; or is suitable for use as an anti-fungal agent, an anti-bacterial agent, or a local anesthetic.

In some embodiments, the active ingredient is or includes Minoxidil, Ketoconazole, Pramoxine Hydrochloride, Lidocaine, a Corticosteroid, a topical antibiotic, Retinoic Acid, or Retinol.

In some embodiments, the active ingredient is or includes Minoxidil; Ketoconazole; Pramoxine Hydrochloride; Lidocaine; a corticosteroid, such as Augmented Betamethasone dipropionate, Clobetasol 17-Propionate, Diflorasone diacetate, Halobetasol propionate, Amcinonide, Betamethasone dipropionate, Betamethasone valerate, Desoximetasone, Diflucortolone valerate, Fluocinonlone acetonide, Fluocinonide, Fluticasone Propionate, Halcinonide, Mometasone furoate, Triamcinolone acetonide, Betamethasone benzoate, Betamethasone dipropionate, Betamethasone valerate, Clocortolone pivalate, Clobetasone 17-Butyrate, Desoximetasone, Fluocinolone acetonide, Flurandrenolide, Fluticasone propionate, Hydrocortisone butyrate, Hydrocortisone valerate, Mometasone furoate, Triamcinolone acetonide, Alclometasone dipropionate, Desonide, Dexamethasone, Dexamethasone sodium Phosphate, Fluocinolone acetonide, Hydrocortisone, Hydrocortisone acetate, Prednicarbate; topical antibiotics, such as Clindamycin, Erythromycin, Tetracycline, Metronidazole, Bacitracin, Bacitracin zinc, neomycin, polymyxin B sulfate, Gentamicin, Mupirocin, Retapamulin; Retinoic Acid; or Retinol.

In some embodiments, a provided composition further comprises a skin-whitening agent. In some embodiments, the skin-whitening agent is hydroquinone, arbutin, kojic acid, bisabolol, azelaic acid, ascorbic acid, magnesium ascorbyl phosphate, cinnamomum subavenium, alpha hydroxy acids, niacinamide, licorice, dihydropinosylvin, 4-benzyl-1,3-benzenediol or pinosylvin.

In some embodiments, a provided composition comprises: (a) about 1% to about 8% (w/w) foam booster; (b) about 0.4% to about 5% (w/w) foaming agent; (c) about 20% to about 98% (w/w) solubilizer; (d) about 0.5% to about 5% (w/w) emollient; and an active ingredient, wherein:
the foam booster is selected from Cocoamidopropyl Betaine, Cocamidopropylamine Oxide, Sodium Lauroyl Sarcosinate, Cetearyl Alcohol, Lauramine Oxide, Laureth-2, Laureth-4, Microcrystalline Cellulose, PEG-150 Distearate, PVP, and combinations thereof;
the foaming agent is selected from one or more Polyoxyethylene Sorbitan Fatty Acid Esters (such as Polysorbate 20, Polysorbate 40, Polysorbate 60, Polysorbate 80, and the like), one or more Polyoxylpropylene-polyoxyethylene Block copolymers (such as Poloxamer 124, Poloxamer 188, Poloxamer 237, Poloxamer 338, Poloxamer 407, and the like), one or more PEG ether of fatty alcohols (such as Brij L9(PEG 9 Lauryl ether), Brij L23(PEG 23 Lauryl ether), and the like), Sodium lauryl Sulphate, Sodium Laureth Sulphate, Disodium Cocoamphodiacetate, Disodium Lauryl Sulfosuccinate, Myristic Acid, Sodium Cocoyl Sarcosinate, Sodium Laureth-2 Sulfate, Sodium Laureth-3 Sulfate, Sodium Lauryl Sulfoacetate, Sucrose Stearate, one or more Polyoxyethylene Hydrogenated Castor Oils (such as Polyoxyl 40 Hydrogenated castor oil, Polyoxyl 60 Hydrogenated castor oil, and the like), and combinations thereof;
the solubilizer is selected from Water, Ethanol, Diethylene Glycol monomethyl ether, Glycerine, Propylene Glycol, Isopropyl alcohol, Polyethylene Glycols, Benzyl alcohol, Dimethyl Sulfoxide, PEG 40 Castor oil, PEG 40 Hydrogenated Castor Oil, PEG 60 Hydrogenated Castor Oil, Sorbitan Stearate, Oleyl Alcohol, and combinations thereof;
the emollient is selected from Dimethicone, Cyclomethicone, Arachis Hypogaea (Peanut) Oil, Canola Oil, Carthamus Tinctorius (Safflower) Seed Oil, Cocos Nucifera (Coconut) Oil, Citrus Aurantium Amara (Bitter Orange) Peel Oil, Citrus Medica Limonum (Lemon) Peel Oil, Citrus Aurantifolia (Lime) Oil, Elaeis Guineensis (Palm) Oil, Glycine Soja (Soybean) Oil, Mentha Piperita (Peppermint) Oil, Olea Europaea (Olive) Oil, PEG-60 Hydrogenated Castor Oil, Papaver Somniferum (Poppy) Seed Oil, Ricinus Communis (Castor) Seed Oil, Sesamum Indicum (Sesame) Seed Oil, Zea Mays (Corn) Oil, Butyrospermum Parkii (Shea) Butter, Theobroma Cacao (Cocoa) Seed Butter, PEG-75 Lanolin, PEG-2 Stearate, PEG-8 Laurate, PPG-15 Stearyl Ether, PPG-20 MethylGlucose Ether Distearate, Lanolin, Lanolin Alcohol, Acetylated Lanolin Alcohol, Lecithin, Caprylic/Capric Triglyceride, Caprylic/Capric/Stearic Triglyceride, Hydrogenated Palm Glycerides, Glycol Stearate, Butylene Glycol, Hexylene Glycol, Propylene Glycol, Cetearyl Alcohol, Cetyl Alcohol, Myristyl Alcohol, Oleyl Alcohol, Stearyl Alcohol, Glyceryl Caprylate, Glyceryl Laurate, Glyceryl Isostearate, Glyceryl Oleate, Glyceryl Stearate, Glyceryl Distearate, Polyglyceryl-3 Oleate, Polyglyceryl-10 Oleate, Isostearic Acid, Lactic Acid, Sodium Lactate, Copernicia Cerifera (Carnauba) Wax, Cera Alba (Beeswax), Euphorbia Cerifera (Candelilla) Wax, Polyquaternium-7, Choleth-24, Methyl Gluceth-10, Methyl Gluceth-20, Diisopropyl Adipate, Methyl Laurate, Cetearyl Ethylhexanoate, Diethyl Sebacate, Ethylhexyl Hydroxystearate, Xanthan Gum, Isopropyl Myristate, Isopropyl Palmitate, Cetyl Palmitate, Octyldodecanol, Acacia, Butyl Stearate, Cholesterol, Cysteine HCl, Lauryl Lactate, Myristyl Lactate, Paraffinum Liquidum (Mineral) Oil, Petrolatum, Polybutene, PVP, Sorbitan Isostearate, Squalane, Tocopherol, Tricaprylin, Urea, Zea Mays (Corn) Starch, Hyaluronic Acid, Sodium Hyaluronate, and combinations thereof; and
the active ingredient is suitable for use in the treatment of alopecia; dermatitis, such as Allergic contact dermatitis, Seborrheic dermatitis, Irritant contact dermatitis, Atopic dermatitis, Neurodermatitis, Perioral dermatitis, Stasis dermatitis, Diaper dermatitis, Lichen simplex chronicus, Dyshidrotic dermatitis, Nummular dermatitis, Autosensitization dermatitis, Spongiotic dermatitis, Lichenoid dermatitis; basal cell carcinoma; acne, such as blackheads, whiteheads, pustules, Acne Vulgaris, Acne Rosacea, Cystic Acne, Nodular Acne, Acne Inversa, Hormonal Acne, Acne conglobata, Acne fulminans, Gram Negative folliculitis, Nodulocystic acne, Pyoderma Faciale, Acne Cosmetica, Pomade Acne, Excoriated Acne, Acne Agminata, Acne excoriee; actinic keratosis, such as Hyperkeratotic actinic keratosis, Pigmented actinic keratosis, Lichenoid actinic keratosis, Atrophic actinic keratosis; rosacea, such as Vascular Rosacea, Acne Rosacea, Pre Rosacea, Papulopustular Rosacea, Late Rosacea, Erythematotelangiectatic Rosacea, Ocular Rosacea, Phymatous Rosacea; or psoriasis, such as Psoriasis vulgaris (Plague Psoriasis), Guttate Psoriasis, Inverse Psoriasis, Pustular Psoriasis, Erythrodermic Psoriasis, Nail Poriasis, Psoriasis of the scalp, Psoriatic Arthritis, Seborrheic Psoriasis, Palmoplantar Psoriasis, skin fold Psoriasis; or is suitable for use as an anti-fungal agent, an anti-bacterial agent, or a local anesthetic.

### Compositions—Additional Components:

In some embodiments, a provided composition further comprises one or more of the following: (f) about 0.02% to about 2.5% (w/w) preservative; (g) about 1% to about 25% (w/w) penetration enhancer; (h) a pH-modifying agent.

In some embodiments, the preservative is selected from Methyl Paraben, Ethyl Paraben, Propyl Paraben, Butyl Paraben, Sodium Methyl Paraben, Sodium Ethyl Paraben, Sodium Propyl Paraben, Phenoxyethanol, Benzyl Alcohol, Chloroxylenol, Imidazolidinyl Urea, Diazolidinyl Urea, Benzoic Acid, Dehydroacetic acid, Sorbic acid, Methylchloroisothiazolinone, Methylisothiazolinone, Sodium Benzoate, Potassium Sorbate, Glyceryl Laurate, Butylated Hydroxy Anisole, Benzalkonium chloride, Benzethonium Chloride, Isobutylparaben, Sodium Butyl Paraben, Phenylpropanol, Dichloro-m-xylenol, Anisic acid, Polyaminopropyl Biguanide, Dimethyloxazolidine, 7-Ethylbicyclooxazolidine, Benzisothiazolonone, Methyldibromo Glutaronitrile, Iodopropynyl Butylcarbamate, Trisodium Ethylenediamine Disuccinate, Tris (hydroxymethyl) nitromethane, Triclosan, Caprylyl Glycol, Ethylhexylglycerin, Sodium Usnate, Sodium Levulinate, Sodium Anisate, Sodium Dehydroacetate, Sodium Salicylate, Sodium Hydroxymethyl Glycinate, Piroctone Olamine, Chlorphenesin, Hexamidine Diisethionate, Glyceryl Undecylenate, and combinations thereof.

In some embodiments, the preservative is suitable for pharmaceutical applications; for example, in some embodiments, the preservative is selected from, at the indicated weight percentage, Methyl Paraben (0.02-0.3%), Ethyl Paraben (0.02-0.3%), Propyl Paraben (0.01-0.6%), Butyl Paraben (0.02-0.4%), Sodium Methyl Paraben (0.02-0.3%), Sodium Ethyl Paraben (0.02-0.3%), Sodium Propyl Paraben (0.01-0.6%), Phenoxyethanol (0.5-1.0%), Benzyl Alcohol (0.01-2.0%), Chloroxylenol (0.1-0.8%), Imidazolidinyl Urea (0.03-0.5%), Diazolidinyl Urea (0.1-0.5%), Benzoic Acid (0.1-0.2%), Sorbic acid (0.05-0.2%), Sodium Benzoate (0.1-0.5%), Potassium Sorbate (0.1-0.2%), Butylated Hydroxy Anisole (0.005-0.02), Benzalkonium chloride (0.002-0.005%), Benzethonium Chloride (0.01-0.02%), and combinations thereof.

In some embodiments, the penetration enhancer is selected from one or more sulfoxides, such as Dimethylsulphoxide (DMSO); one or more Azones, such as Laurocapram; one or more pyrrolidones (pyrrolidinones), such as 2-pyrrolidone and N-methyl-2-pyrrolidone); one or more Alcohols (Alkanols), such as Ethanol (“alcohol”), Decanol, Lauryl alcohol; one or more Glycols, such as Propylene Glycol, Diethylene Glycol, tetraethylene glycol; one or more Fatty acids, such as lauric acid, myristic acid, capric acid; one or more Non-ionic surfactants, such as polyoxyethylene-2-oleyl ether, polyoxyethylene-2-stearyl ether; one or more Essential oils, such as eucalyptus, chenopodium, ylang-ylang, L-menthol; Isopropyl palmitate; Isopropyl myristate; Glyceryl mono laurate; Oleic acid; 1,3-Butylene glycol; Lecithin; Dipropylene glycol; Glyceryl monooleate; Methyl laurate; Padimate O; Octyl salicylate; Urea; Sesquiterpene; Dimethyl formamide, and combinations thereof.

In some embodiments, the pH-modifying agent is selected from hydrochloric acid, sodium hydroxide, phosphoric acid, acid phthalate buffer, neutralised phthalate buffer, phosphate buffer, alkaline borate buffer, acetate buffer, citric acid, sodium citrate buffer, arginine, glutamic acid, glycine, tromethamine, 2-aminobutanol, DL-2-pyrrolidone-5-carboxylic acid, aminomethylpropanediol, 2-aminoethanesulfonic acid, triethanolamine, and combinations thereof.

In some embodiments, in a provided composition: (a) the foam booster consists essentially of about 3% to about 6% (w/w) cocoamidopropyl betaine; (b) the foaming agent consists essentially of about 0.2% to about 1.5% (w/w) polysorbate 80 and about 1.5% to about 3.5% (w/w) polyoxyethylene-polyoxypropylene copolymer; (c) the solubilizer consists essentially of about 5% to about 10% (w/w) ethyl alcohol (95%) and about 6% to about 10% (w/w) propylene glycol; and (d) the emollient consists essentially of about 1% to about 2% (w/w) PEG 75 lanolin; and the composition comprises balance water; and the composition optionally further comprises one or more of the following: (e) an active ingredient suitable for use in the treatment of alopecia, dermatitis, basal cell carcinoma, acne, actinic keratosis, rosacea, or psoriasis, or is suitable for use as an anti-fungal agent, an anti-bacterial agent, or a local anesthetic; (f) about 0.02% to about 2.5% (w/w) preservative; (g) about 1% to about 25% (w/w) penetration enhancer; (h) a pH-modifying agent.

In some embodiments, in a provided composition: (a) the foam booster consists essentially of about 1% to about 2% (w/w) cocoamidopropylamine oxide; (b) the foaming agent consists essentially of about 0.2% to about 1.5% (w/w) polysorbate 80 and about 0.2% to about 0.4% (w/w) polyoxyethylene-polyoxypropylene copolymer; (c) the solubilizer consists essentially of about 5% to about 10% (w/w) ethyl alcohol (95%) and about 10% to about 20% (w/w) propylene glycol; and (d) the emollient consists essentially of about 1% to about 2% (w/w) PEG 6 caprylic/capric glyceride; and the composition comprises balance water; and the composition optionally further comprises one or more of the following: (e) an active ingredient suitable for use in the treatment of alopecia, dermatitis, basal cell carcinoma, acne, actinic keratosis, rosacea, or psoriasis, or is suitable for use as an anti-fungal agent, an anti-bacterial agent, or a local anesthetic; (f) about 0.02% to about 2.5% (w/w) preservative; (g) about 1% to about 25% (w/w) penetration enhancer; (h) a pH-modifying agent.

In some embodiments, in a provided composition: (a) the foam booster consists essentially of about 3% to about 7% (w/w) sodium lauroyl sarcosinate; (b) the foaming agent consists essentially of about 1% to about 2% (w/w) PEG 40 hydrogenated castor oil and about 1.0% to about 2.5% (w/w) polyoxy lauryl ether; (c) the solubilizer consists essentially of about 5% to about 10% (w/w) ethyl alcohol (95%) and about 10% to about 20% (w/w) propylene glycol; and (d) the emollient consists essentially of about 0.5% to about 1% (w/w) sodium salt of pyrrolidone carbonic acid; and the composition comprises balance water; and the composition optionally further comprises one or more of the following: (e) an active ingredient suitable for use in the treatment of alopecia, dermatitis, basal cell carcinoma, acne, actinic keratosis, rosacea, or psoriasis, or is suitable for use as an anti-fungal agent, an anti-bacterial agent, or a local anesthetic; (f) about 0.02% to about 2.5% (w/w) preservative; (g) about 1% to about 25% (w/w) penetration enhancer; (h) a pH-modifying agent.

In some embodiments, a provided composition consists essentially of: about 1.0% to about 2.0%, e.g., about 1.5%, (w/w) Cocamidopropyl Betaine; about 1.0% to about 3.0%, e.g., about 2.0%, (w/w) Polysorbate 80; about 1.0% to about 3.0%, e.g., about 2.0%, (w/w) Polyoxylpropylene-polyoxylethylene Block Copolymer; about 5.0% to about 10.0%, e.g., about 7.5%, (w/w) Propylene Glycol; about 6.0% to about 10.0%, e.g., about 8.0%, (w/w) Ethyl Alcohol; about 0.5% to about 1.5%, e.g., about 1.0%, (w/w) PEG-75 Lanolin; about 1.0% to about 2.0%, e.g., about 1.5%, (w/w) Magnesium Ascorbyl Phosphate; about 2.0% to about 4.0%, e.g., about 3.0%, (w/w) Arbutin; and balance water.

In some embodiments, a provided composition consists essentially of: about 1.0% to about 4.0%, e.g., about 2.5%, (w/w) Sodium Lauroyl Sarcosinate; about 3.0% to about 5.0%, e.g., about 4.0%, (w/w) Polyoxylpropylene-polyoxylethylene Copolymer; about 25.0% to about 35.0%, e.g., about 30.0%, (w/w) Propylene Glycol; about 2.0% to about 8.0%, e.g., about 5.0%, (w/w) Ethyl Alcohol; about 0.01% to about 0.1%, e.g., about 0.05%, (w/w) Citric Acid; about 0.1% to about 1.0%, e.g., about 0.5%, (w/w) Lactic Acid; about 2.0% to about 5.0% (w/w) Minoxidil; about 1.0% to about 3.0%, e.g., about 2.0%, (w/w) Diethylene Glycol Monoethyl Ether; about 1.0% to about 2.0%, e.g., about 1.5%, (w/w) Polyoxy lauryl Ether; about 0.01% to about 0.1%, e.g., about 0.05%, (w/w) Benzyl Alcohol; and balance water.

In some embodiments, a provided composition consists essentially of: about 1.0% to about 2.0%, e.g., about 1.5%, (w/w) Cocamidopropylamine Oxide; about 0.1% to about 1.0%, e.g., about 0.5%, (w/w) PEG 40 Hydrogenated Castor Oil; about 6.0% to about 10.0%, e.g., about 8.0%, (w/w) Propylene Glycol; about 5.0% to about 10.0%, e.g., about 7.5%, (w/w) Isopropyl Alcohol; about 1.0% to about 2.0%, e.g., about 1.5%, (w/w) PEG-6 Caprylic/Capric Glyceride; about 0.05% to about 0.10% (w/w) Clobetasol Propionate; and balance water.

### EXAMPLES

General procedure for making compositions. Supplied with the constituent makeup, as described herein, making a provided composition is well within the knowledge and skill of those skilled in the art using customary methods.

In some embodiments, a solvent or combination of solvents is provided, and active ingredient (if present) is dissolved or suspended in the solvent(s). The primary and secondary surfactant/foaming agents are incorporated. If active ingredient is present, they can aid dissolution of an active ingredient if necessary based on solubility. The foam booster(s), along with any emollient(s), preservative(s), and pH adjuster(s), if present, are added. The desired batch size is made up with the required quantity of water or vehicle.

In each of Examples 1-3, a composition according to the present invention was prepared according to the procedure above, and having the indicated composition. Foam from each composition was then evaluated as follows.

An F3 L11 200/100 mesh pump was used in the foam evaluation. F3 depicts the design of the pump, L11 indicates 0.75 mL output per stroke with liquid:air ratio of 1:11, and the foam is dispensed through a net having mesh size of 200/100.

Each composition was dispensed through the pump by actuating the pump five times, and the resulting foam was collected in a graduated tared beaker. The volume of the 5 actuations and the weight of the foam were measured to calculate the density of the foam (the volume of 5 actuations in each case was about 40 mL). The foam was then allowed to subside, and the time for the foam to dissipate into liquid was measured with a stopwatch.

### Example 1

Composition

| Ingredient | Function | Amount (% w/w) |
|---|---|---|
| ethyl alcohol 95% | solubilizer | 7.0 |
| propylene glycol | solubilizer | 8.0 |
| polysorbate 80 | foaming agent | 1.0 |
| polyoxyethylene- | foaming agent | 2.0 |
| polyoxypropylene copolymer | | |
| cocoamidopropyl betaine | foam booster | 4 |
| PEG 75 lanolin | emollient | 1.5 |
| purified water | vehicle | q.s. |

| | |
|---|---|
| specific gravity | 1.005 g/mL |

Resulting Foam

| | |
|---|---|
| density of foam | 0.095 g/mL |
| dissipation time from | about 47 min |
| foam to liquid | |

### Example 2

Composition

| Ingredient | Function | Amount (% w/w) |
|---|---|---|
| ethyl alcohol 95% | solubilizer | 8 |
| propylene glycol | solubilizer | 15 |
| polysorbate 80 | foaming agent | 0.8 |
| polyoxyethylene- | foaming agent | 0.3 |
| polyoxypropylene copolymer | | |
| cocoamidopropylamine oxide | foam booster | 1.2 |
| PEG 6 caprylic capric glyceride | emollient | 1.4 |
| purified water | vehicle | q.s. |

| | |
|---|---|
| specific gravity | 0.970 g/mL |

Resulting Foam

| | |
|---|---|
| density of foam | 0.0935 g/mL |
| dissipation time from | about 49 min |
| foam to liquid | |

### Example 3

Composition

| Ingredient | Function | Amount (% w/w) |
|---|---|---|
| ethyl alcohol 95% | solubilizer | 7.5 |
| propylene glycol | solubilizer | 12.0 |
| PEG 40 hydrogenated castor oil | foaming agent | 1.5 |
| polyoxy lauryl ether | foaming agent | 2.0 |
| sodium lauroyl sarcosinate | foam booster | 3.5 |
| sodium salt of pyrrolidone | emollient | 0.8 |
| carbonic acid | | |
| purified water | vehicle | q.s. |

| | |
|---|---|
| specific gravity | 0.985 g/mL |

Resulting Foam

| | | |
|---|---|---|
| density of foam | 0.0952 | g/mL |
| dissipation time from | ^{~}60 | min |
| foam to liquid | | |

What is claimed is:

1. A liquid composition suitable for topical administration and for use with a non-aerosol pump, wherein actuation of the pump produces a foam, the composition comprising:
(a) about 1% to about 8% (w/w) foam booster;
(b) about 0.4% to about 5% (w/w) foaming agent; and
(c) about 20% to about 98% (w/w) solubilizer;
wherein the composition is substantially free of propellant.

2. The liquid composition of claim 1, characterized in that the density of the foam produced is about 0.076 to about 0.102 g/mL.

3. The liquid composition of claim 1, characterized in that the time for the foam produced to dissipate into a liquid is about 45 minutes or longer for a volume of about 40 mL of foam.

4. The liquid composition of claim 1, characterized in that the specific gravity of the composition is about 0.85 to about 1.20 g/mL.

5. The composition of any of claims 1-4, comprising:
(a) about 3% to about 6% (w/w) foam booster;
(b) about 1.7% to about 5% (w/w) foaming agent;
(c) about 5% to about 30% (w/w) solubilizer other than water;
and balance water.

6. The composition of any of claims 1-4, comprising:
(a) about 1% to about 2% (w/w) foam booster;
(b) about 0.4% to about 1.9% (w/w) foaming agent;
(c) about 5% to about 30% (w/w) solubilizer other than water;
and balance water.

7. The composition of any of claims 1-4, comprising:
(a) about 3% to about 7% (w/w) foam booster;
(b) about 2% to about 4.5% (w/w) foaming agent;
(c) about 5% to about 30% (w/w) solubilizer other than water;
and balance water.

8. The composition of any of claims 1-4, comprising:
(a) about 1.5% to about 6% (w/w) foam booster;
(b) about 1.5% to about 5.0% (w/w) foaming agent;
(c) about 10% to about 20% (w/w) solubilizer other than water;
and balance water.

9. The composition of any of claims 1-4, comprising:
(a) about 2.0% to about 7.5% (w/w) foam booster;
(b) about 2.0% to about 5.0% (w/w) foaming agent;
(c) about 25% to about 45% (w/w) solubilizer other than water;
and balance water.

10. The composition of any of claims 1-4, comprising:
(a) about 1.0% to about 2.0% (w/w) foam booster;
(b) about 0.5% to about 2.0% (w/w) foaming agent;
(c) about 10% to about 20% (w/w) solubilizer other than water;
and balance water.

11. The composition of any of claims 1-10, wherein the foam booster is selected from the group consisting of Cocoamidopropyl Betaine, Cocamidopropylamine Oxide, Sodium Lauroyl Sarcosinate, Cetearyl Alcohol, Lauramine Oxide, Laureth-2, Laureth-4, Microcrystalline Cellulose, PEG-150 Distearate, PVP, Coco dimethyl carboxymethyl betaine, Cocoamidopropyl Hydroxysultaine, Cocamide Monoethanolamide, Cocamidoethyl Betaine, Lauryl Amidopropyl Betaine, Oleyl betaine, Lauryl Dimethyl Carboxymethyl Betaine, Cocamide Ethyl Betaine, Cocamide oxide, Lauryl Betaine, Lauryl Hydroxy Sultaine, Lauryl Sultaine, Ammonium Myreth Sulfate, Avocadamide DEA, Avocadoamide DIPA, Caprylyl Pyrrolidone, Cocamide DEA, Cocamide DIPA, Cocamide MEA, Dilinoleyl Amidopropyl Trimonium Chloride, Gynostemma Pentaphyllum Leaf Extract, Hydrogenated Coconut Acid, Hydrolyzed Keratin, Lauramide DEA, Lauramide MEA, Lauryl Myristyl Amine Oxide, Lauryl Pyrrolidone, Lauryldimonium Hydroxypropyl Cocoglucosides Chloride, Linoleamide DEA, Myristamine Oxide, PEG-2 Cocamide, PEG-3 Glyceryl Cocoate, PEG-30 Glyceryl Soyate, PEG-7 Glyceryl Soyate, PEG-80 Glyceryl Cocoate, Polyquaternium-43, Potassium Soyate, PPG-2 Hydroxyethyl Coco Isostearamide, Sesamide DIPA, Sodium C12-15 Pareth Sulfate, Sodium C14-16 Olefin Sulfonate, Sodium Cocoamphoacetate, Sodium Methylpalmitoyl Taurate, Sodium Methylstearoyl Taurate, Sodium Stearoyl Lactylate, Sodium Tridecyl Sulfate, Sodium Olivamphoacetate, and combinations thereof.

12. The composition of any of claims 1-10, wherein the foaming agent is selected from the group consisting of one or more Polyoxyethylene Sorbitan Fatty Acid Esters, one or more Polyoxylpropylene-polyoxyethylene Block copolymers, one or more PEG ether of fatty alcohols, Sodium lauryl Sulphate, Sodium Laureth Sulphate, Disodium Cocoamphodiacetate, Disodium Lauryl Sulfosuccinate, Myristic Acid, Sodium Cocoyl Sarcosinate, Sodium Laureth-2 Sulfate, Sodium Laureth-3 Sulfate, Sodium Lauryl Sulfoacetate, Sucrose Stearate, one or more Polyoxyethylene Hydrogenated Castor Oils, Polyoxyl Lauryl Ether, PEG Ether of Lauryl Alcohol, Ammonium Laureth-2 Sulfate, Ammonium Lauryl Sulfosuccinate, Capramide DEA, Cocamidopropyl Dimethylaminohydroxypropyl Hydrolyzed Collagen, Cocoyl Hydrolyzed Soy Protein, Dihydroxyethyl Soyamine Dioleate, Dilinoleamidopropyl Dibetaine, Disodium Cocoyl Glutamate, Disodium PEG-5 Laurylcitrate Sulfosuccinate, Hydroxypropyl Guar, Laureth-1 Phosphate, Lauryl Betaine, Lauryl Phosphate, Oleoyl Sarcosine, PEG-7 Glyceryl Cocoate, Potassium Cocoyl Glycinate, Potassium Cocoyl Hydrolyzed Collagen, Potassium Lauroyl Sarcosinate, Potassium Lauryl Sulfate, Ricinoleamidopropyl Betaine, Sodium C12-13 Pareth-8 Carboxylate, Sodium Cetyl Sulfate, Sodium Cocamphoacetate, Sodium Cocomonoglyceride Sulfate, Sodium Coco-sulfate, Sodium Hydroxypropylphosphate Laurylglucoside Crosspolymer, Sodium Hydroxypropylsulfonate Cocoglucoside Crosspolymer, Sodium Hydroxypropylsulfonate Decylglucoside Crosspolymer, Sodium Lauramphoacetate, Sodium Lauroyl Glutamate, Sodium Lauroyl Hydrolyzed Silk, Sodium Lauroyl Lactylate, Sodium Lauroyl Methyl Isethionate, Sodium Lauroyl Methylaminopropionate, Sodium Lauryl Phosphate, Sodium Laurylglucoside Hydroxypropyl Sulfonate, Sodium Methyl Lauroyl Taurate, Sodium Methylcocoyl Taurate, Sodium Methylmyristoyl Taurate, Sodium Myristate, Sodium Myristoyl Glutamate, Sodium Myristoyl Sarcosinate, Sodium Myristyl Sulfate, Sodium Palmitoyl Sarcosinate, Sodium PEG-7 Olive Oil Carboxylate, Sodium Stearoyl Glutamate, Sodium Trideceth Sulfate, Sodium Trideceth-4 Carboxylate, Sodium Trideceth-7 Carboxylate, Sucrose Cocoate, TEA-Dodecyl benzenesulfonate, TEA-Cocoyl Glutamate, TEA-Cocoyl Sarcosinate, TEA-Laureth Sulfate, Trideceth-4 Carboxylic Acid, Trideceth-8 Carboxylic Acid, Yogurt Powder, Yucca Schidigera Extract and combinations thereof.

13. The composition of any of claims 1-10, wherein the solubilizer is selected from the group consisting of Water, Ethanol, Diethylene Glycol monomethyl ether, Glycerine, Propylene Glycol, Isopropyl alcohol, Polyethylene Glycols, Benzyl alcohol, Dimethyl Sulfoxide, PEG 40 Castor oil, PEG 40 Hydrogenated Castor Oil, PEG 60 Hydrogenated Castor Oil, Sorbitan Stearate, Oleyl Alcohol, Dimethylformamide, and combinations thereof.

14. The composition of any of claims 1-13, further comprising:
(d) about 0.5% to about 5% (w/w) emollient.

15. The composition of claim 5, further comprising:
(d) about 1% to about 2% (w/w) emollient.

16. The composition of claim 6, further comprising:
(d) about 1% to about 2% (w/w) emollient.

17. The composition of claim 7, further comprising:
(d) about 0.5% to about 1% (w/w) emollient.

18. The composition of claim 8, further comprising:
(d) about 1.0% to about 2.0% (w/w) emollient.

19. The composition of claim 9, further comprising:
(d) about 0.5% to about 1.0% (w/w) emollient.

20. The composition of claim 10, further comprising:
(d) about 1.0% to about 5.0% (w/w) emollient.

21. The composition of any of claims 14-20, wherein the emollient is selected from the group consisting of Dimethicone, Cyclomethicone, Arachis Hypogaea (Peanut) Oil, Canola Oil, Carthamus Tinctorius (Safflower) Seed Oil, Cocos Nucifera (Coconut) Oil, Citrus Aurantium Amara (Bitter Orange) Peel Oil, Citrus Medica Limonum (Lemon) Peel Oil, Citrus Aurantifolia (Lime) Oil, Elaeis Guineensis (Palm) Oil, Glycine Soja (Soybean) Oil, Mentha Piperita (Peppermint) Oil, Olea Europaea (Olive) Oil, PEG-60 Hydrogenated Castor Oil, Papaver Somniferum (Poppy) Seed Oil, Ricinus Communis (Castor) Seed Oil, Sesamum Indicum (Sesame) Seed Oil, Zea Mays (Corn) Oil, Butyrospermum Parkii (Shea) Butter, Theobroma Cacao (Cocoa) Seed Butter, PEG-75 Lanolin, PEG-2 Stearate, PEG-8 Laurate, PPG-15 Stearyl Ether, PPG-20 MethylGlucose Ether Distearate, Lanolin, Lanolin Alcohol, Acetylated Lanolin Alcohol, Lecithin, Caprylic/Capric Triglyceride, Caprylic/Capric/Stearic Triglyceride, Hydrogenated Palm Glycerides, Glycol Stearate, Butylene Glycol, Hexylene Glycol, Propylene Glycol, Cetearyl Alcohol, Cetyl Alcohol, Myristyl Alcohol, Oleyl Alcohol, Stearyl Alcohol, Glyceryl Caprylate, Glyceryl Laurate, Glyceryl Isostearate, Glyceryl Oleate, Glyceryl Stearate, Glyceryl Distearate, Polyglyceryl-3 Oleate, Polyglyceryl-10 Oleate, Isostearic Acid, Lactic Acid, Sodium Lactate, Copernicia Cerifera (Carnauba) Wax, Cera Alba (Beeswax), Euphorbia Cerifera (Candelilla) Wax, Polyquaternium-7, Choleth-24, Methyl Gluceth-10, Methyl Gluceth-20, Diisopropyl Adipate, Methyl Laurate, Cetearyl Ethylhexanoate, Diethyl Sebacate, Ethylhexyl Hydroxystearate, Xanthan Gum, Isopropyl Myristate, Isopropyl Palmitate, Cetyl Palmitate, Octyldodecanol, Acacia, Butyl Stearate, Cholesterol, Cysteine HCl, Lauryl Lactate, Myristyl Lactate, Paraffinum Liquidum (Mineral) Oil, Petrolatum, Polybutene, PVP, Sorbitan Isostearate, Squalane, Tocopherol, Tricaprylin, Urea, Zea Mays (Corn) Starch, Hyaluronic Acid, Sodium Hyaluronate, PEG 90 Diisostearte, PEG/PPG-8/3 Diisostearate, C₁₂₋₁₅ Alkyl Benzoate, Cetyl Ricinoleate Benzoate, Ethylhexyl Hydroxystearate Benzoate, Methyl Gluceth-20 Benzoate, Dipropylene Glycol Dibenzoate, Dimethicone PEG/PPG-20/23 Benzoate, Dimethicone PEG-8 Benzoate, Dipropylene Glycol Dibenzoate, PPG-15 Stearyl Ether Benzoate, Bis-PEG/PPG-20/20 dimethicone, Stearoxy Dimethicone, Behenoxy Dimethicone, Stearyl Dimethicone, Cetyl Dimethicone, Cerotyl Dimethicone, PEG-8 Dimethicone, PEG-9 Dimethicone, PEG-14 Dimethicone, PEG-12 Dimethicone/PPG-20 Crosspolymer, Undecylcrylene Dimethicone, Perfluorononyl Dimethicone, PEG-8 PG-Coco-Glucoside Dimethicone, Dimethicone/Vinyl Dimethicone Crosspolymer, Dimethicone PEG-8 Beeswax, C₂₆₋₂₈ Alkyl Dimethicone, Caprylyl Methicone, Phenyl Trimethicone, C₂₆₋₂₈ Alkyl Methicone, Amaranthus Caudatus (Amaranth) Seed Oil, Adansonia Digitata (Baobab) Seed Oil, Avena Sativa (Oat) Kernel Oil, Actinidia Chinensis (Kiwi) Seed Oil, Aleurites Moluccana (Kukui nut) Seed Oil, Argania Spinosa (Argan) Kernel Oil, Borago Officinalis (Borage) Seed Oil, Bertholetta Excelsa (Brazil Nut) Seed Oil, Brassica Oleracea Italica (Broccoli) Seed Oil, Brassica Campestris (Rapeseed) Seed Oil, Carapa Guaianensis (Andiroba) Seed Oil, Corylus Avellana (Hazel) Seed Oil, Cannabis Sativa (Hemp) Seed Oil, Camelina Sativa (Camelina) Seed Oil, Camellia Oleifera (Tea) Seed Oil, Crambe Abyssinica Seed Oil, Citrus Paradisi (Grapefruit) Seed Oil, Cucurbita Pepo (Pumpkin) Seed Oil, Cucumis Sativus (Cucumber) Seed Oil, Citrullus Lanatus (Watermelon) Seed Oil, Carica Papaya (Papaya) Seed Oil, Eucalyptus Globulus Leaf Oil, Euterpe Oleracea (Acai) Fruit Oil, Emu Oil, Fragaria Ananassa (Strawberry) Seed Oil, Hippophae Rhamnoides (Seabuckthorn) Oil, Helianthus Annuus (Sunflower) Seed Oil, Juglans Regia (Walnut) Seed Oil, Limnanthes Alba (Meadowfoam) Seed Oil, Linum Usitatissimum (Linseed) Seed Oil, Macadamia Integrifolia (Macadamia) Seed Oil, Mangifera Indica (Mango) Seed Oil, Mauritia Flexuosa Fruit Oil, Moringa Pterygosperma (Moringa) Seed Oil, Nigella Sativa (Cumin) Seed Oil, Neatsfoot Oil, Orbignya Oleifera (Babassu) Seed Oil, Oenothera Biennis (Evening Primrose) Oil, Oryza Sativa (Rice) Bran Oil, Olus Oil, Passiflora Edulis (Passion) Seed Oil, Passiflora Incarnata Seed Oil, Pistacia Vera (Pistachio) Seed Oil, Prunus Amygdalus Dulcis (Sweet Almond) Oil, Prunus Armeniaca (Apricot) Kernel Oil, Prunus Domestica (Plum) Seed Oil, Prunus Avium (Sweet Cherry) Seed Oil, Persea Gratissima (Avocado) Oil, Punica Granatum (Pomegranate) Seed Oil, Prunus Persica (Peach) Kernel Oil, Ribes Nigrum (Black Currant) Seed Oil, Rose Canina (Rose Hips) Fruit Oil, Rosa Moschata Seed Oil, Rubus Occidentalis (Black Raspberry) Seed Oil, Rubus Idaeus (Raspberry) Seed Oil, Salvia Hispanica (Chia) Seed Oil, Scierocarya Birrea (Marula) Kernel Oil, Silybum Marianum (Milk Thistle) Seed Oil, Simmondsia Chinensis (Jojoba) Seed Oil, Sisymbrium Irio (Rocket) Seed Oil, Solanum Lycopersicum (Tomato) Seed Oil, Triticum Vulgare (Wheat) Germ Oil, Vaccinium Corymbosum (Blueberry) Seed Oil, Vaccinium Macrocarpan (Cranberry) Seed Oil, Vaccinium Myrtillus (Bilberry) Seed Oil, Vitis Vinifera (Grape) Seed Oil, Algae Extract, Aloe Barbadensis Leaf Extract, Althaea Officinalis Extract, Anthemis Nobilis Flower Extract, Arnica Montana Flower Extract, Ascophyllum Nodosum Extract, Avena Sativa (Oat) Kernel Extract, Caviar Extract, Centella Asiatica Extract, Cocos Nucifera (Coconut) Extract, Coffea Arabica (Coffee) Extract, Cymbopogon Citratus Extract, Daucus Carota Sativa (Carrot) Extract, Echinacea Angustifolia Extract, Echinacea Purpurea Extract, Fucus Vesiculosus Extract, Hydrolyzed Adansonia Digitata Extract, Lactobacillus/Phoenix Dactylifera (Date) Fruit Ferment Extract, Linum Usitatissimum (Linseed) Seed Extract, Pisum Sativum (Pea) Extract, Pollen Extract, Prunus Amygdalus Dulcis (Sweet Almond) Fruit Extract, Prunus Armeniaca (Apricot) Fruit Extract, Pyrus Malus (Apple) Fruit Extract, Sargassum Vulgare Extract, Sea Cucumber Extract, Shorea Robusta Leaf Extract, Stevia Rebaudiana Extract, Symphytum Officinale Leaf Extract, Urtica Dioica (Nettle) Extract, Vaccinium Angustifolium (Blueberry) Fruit Extract, Vaccinium Macrocarpan (Cranberry) Fruit Extract, Vaccinium Myrtillus Extract, Astrocaryum Murumuru Seed Butter, Astrocaryum Tucuma Seed Butter, Elaeis Guineensis (Palm) Butter, Garcini Indica Seed Butter, Irvingia Gabonensis Kernel Butter, Mangifera Indica (Mango) Seed Butter, Shorea Stenoptera Butter, Shorea Stenoptera Seed Butter, Theobroma Grandiflorum Seed Butter, Almond Oil PEG-8 Esters, one or more Babassu Oil Glycereth-8 Esters, one or more Coconut Oil Glycereth-8 Esters, one or more Cottonseed Oil Glycereth-8 Esters, one or more Grape Seed Oil PEG-8 Esters, one or more Cannabis Sativa Seed Oil PEG-8 Esters, one or more Macadamia Seed Oil Glycereth-8 Esters, one or more Peach Kernel Oil Glycereth-8 Esters, one or more Avocado Oil Glycereth-8 Esters, one or more Sesame Oil Glycereth-8 Esters, one or more Soybean Oil Glycereth-8 Esters, one or more Sunflower Seed Oil Glycereth-8 Esters, one or more Olive Oil PEG-7 Esters, one or more PEG-6 Capric Glycerides, one or more PEG-6 Caprylic/Capric Glycerides, one or more PEG-6 Caprylic/Capric Triglycerides, PEG-3 Glyceryl Cocoate, PEG-7 Glyceryl Cocoate, PEG-30 Glyceryl Cocoate, PEG-80 Glyceryl Cocoate, PEG-10 Lanolin, PEG-20 Lanolin, PEG-30 Lanolin, PEG-60 Lanolin, PEG-8 Distearate, PEG-90 Diisostearate, PEG/PPG-8/3 Diisostearate, PEG-8 Dilaurate, PEG-12 Laurate, PEG-12 Dilaurate, PEG/PPG-8/3 Laurate, PEG-5 Isononanoate, PEG-2 Diisononanoate, PEG-2 Dioctanoate, PEG-40 Butyloctanol, one or more PEG-10 Sunflower Glycerides, one or more PEG-16 Macadamia Glycerides, PEG-8 Dioleate, one or more PEG-75 Shea Butter Glycerides, one or more PEG-70 Mango Glycerides, PEG-5 Soy Sterol, PEG-25 PABA, PEG-35 Castor Oil, PPG-2 Myristyl Ether Propionate, PPG-3 Myristyl Ether, PPG-5-Ceteth-20, PPG-5 Pentaerythrityl Ether, PPG-10 Cetyl Ether, PPG-10 MethylGlucose Ether, PPG-11 Stearyl Ether, PPG-14 Butyl Ether, PPG-20 MethylGlucose Ether, Lanolin Oil, Lanolin Wax, Acetylated Lanolin, Hydroxylated Lecithin, Capric/Capric Triglyceride, Caprylic/Capric/Lauric Triglyceride, Caprylic/Capric/Linoleic Triglyceride, one or more Hydrogenated Coco-Glycerides, one or more Olive glycerides, one or more Soybean Glycerides, one or more Wheat Germ Glycerides, Glycol Distearate, Glycol Palmitate, Caprylyl Glycol, Butylene Glycol Cocoate, Butylene Glycol Dicaprylate/Dicaprate, Propylene Glycol Heptanoate, Propylene Glycol Caprylate, Propylene Glycol Dicaprate, Propylene Glycol Dicaprylate/Dicaprate, Propylene Glycol Diethylhexanoate, Propylene Glycol Stearate, Neopentyl Glycol Diheptanoate, Neopentyl Glycol Dicaprate/Dicaprylate, Neopentyl Glycol Dioctanoate, Batyl Alcohol, Behenyl Alcohol, Chimyl Alcohol, Lauryl Alcohol, Glyceryl Caprylate/Caprate, Glyceryl Dilaurate, Glyceryl Triacetyl Hydroxystearate, Glyceryl Undecylenate, Oleyl Glyceryl Ether, Bis-Diglyceryl Polyacyladipate-1,Bis-Diglyceryl Polyacyladipate-2, Polyglyceryl-2 Triisostearate, Polyglyceryl-3 Caprate, Polyglyceryl-3 Laurate, Polyglyceryl-3 Stearate, Polyglyceryl-3 Polyricinoleate, Polyglyceryl-4 Caprate, Polyglyceryl-6 Octastearate, Polyglyceryl-6 Dioleate, Polyglyceryl-10 Decaoleate, Polyglyceryl-10 Decaisostearate, Polyglyceryl-10 Decamacadamiate, Polyglyceryl-10 Heptaoleate, Polyglyceryl-10 Mono/Dioleate, Polyglyceryl-10 Pentastearate, Polyglyceryl-10 Tetraoleate, Diisostearoyl Polyglyceryl-3 Dimer Diinoleate, Triisostearyl Polyglycerol-3 Dimer Dilinoleate, Arachidonic Acid, Coconut Acid, Isostearyl Linoleate Abietic Acid, Linoleic Acid, Linolenic Acid, Salicylic Acid, Sodium C₁₂₋₁₅ Alkoxypropyl iminodipropionate, Sodium Lauroyl Lactylate, Sodium Stearoyl Lactylate, Sodium Usnate, Disodium Oleamido MEA-Sulfosuccinate, Sodium Acrylates Copolymer, one or more Jojoba Wax PEG-80 Esters, Citrus Medica Limonum (Lemon) Peel Wax, Narcissus Poeticus Wax, Citrus Aurantium Dulcis (Orange) Peel Wax, Castor Isostearate Beeswax Succinate, Polyquaternium-6, Polyquaternium-37, Glycereth-26, Laureth-12 Succinate, Ceteareth-20, Ceteareth-22, Ceteareth-6 Olivate, Trideceth-6, Trideceth-9, Ceteth-24, Myreth-3 Myristate, Dihydrocholeth-30, one or more Shea Butter Glycereth-8 Esters, Cetyl Acetate, Tocopheryl Acetate, Panthenyl Triacetate, Dibutyl Adipate, Diisodecyl Adipate, Dioctyl Adipate, Methyl Caprate, Methyl Cocoate, Methyl Lactate, Methyl Myristate, Methyl Oleate, Methyl Palmitate, Methyl Soyate, Polymethylsilsesquioxane, Methylglucose Dioleate, Trimethylolpropane Triisostearate, C₁₂₋₁₅ Alkyl Ethylhexanoate, Cetyl Ethylhexanoate, Diethylhexyl 2,6-naphtha late, Diethylhexyl Carbonate, Diethylhexyl Maleate, Ethyl Macadamiate, Ethyl Linoleate, Ethyl Olivate, Ethylhexyl Ethylhexanoate, Ethylhexyl Isononanoate, Ethylhexyl Pelargonate, Ethylhexyl Palmitate, Ethylhexyl Stearate, Triethylhexanoin, Sclerotium Gum, Isopropyl Linoleate, Diisopropyl Sebacate, Guar Hydroxypropyltrimonium Chloride, Hydroxypropyl Guar Hydroxypropyltrimonium Chloride, Hydroxypropyl Guar, Borageamidopropyl Phosphatidyl-PG-Dimonium Chloride, Hydroxypropyltrimonium Hydrolyzed Wheat Protein, C₃₀₋₃₈ Olefin/Isopropyl Maleate/MA Copolymer, Hydroxypropyl Oxidized Starch PG-Trimonium Chloride, Starch Hydroxypropyltrimonium Chloride, Isopropyl C₁₂₋₁₅-Pareth-9-Carboxylate, Cetyl Ricinoleate, Isocetyl Myristate, Isocetyl Palmitate, Isocetyl Stearate, Tricetyl Phosphate, Stearyl Heptanoate, Distearyl Ether, Isostearyl Neopentanoate, Isostearyl Avocadate, Isostearyl Isostearate, Isostearyl Palmitate, Diisostearyl Malate, Diisostearyl Dimer Dilinoleate, Diisostearyl Fumarate, Diisostearyl Malate, Triisostearyl Citrate, Triisostearyl Trilinoleate, Octyl Stearate, Octyl Isononanoate, Octyl Palmitate, Octyl Cocoate, Octyl Palmitate, Octyldodecyl PPG-3 Myristyl Ether Dimer Dilinoleate, Octyldodecyl Neodecanoate, Octyldodecyl Myristate, Octyl Methoxycinnamate, Octyldodecyl Ricinoleate, Dioctyl Sebacate, Dioctyldodecyl Fluoroheptyl Citrate, Trioctyldodecyl Citrate, Butyloctyl Salicylate, 1,2-Hexanediol, AMP-Isostearoyl Hydrolyzed Collagen, Arachidyl Behenate, one or more Avocado Sterols, Benzophenone-3,Benzyl Nicotinate, Bisabolol, Butyl Myristate, Castor Isostearate Succinate, Castoryl Maleate, Ceramide 3, Cetearyl Isononanoate, Cholesteryl Stearate, Cholesteryl/Octydodecyl Lauroyl Glutamate, Cocamide MEA, Cyclopentasiloxane, Cyclotetrasiloxane, Decyl Cocoate, Decyl Oleate, Dicaprylyl Carbonate, Dicaprylyl Ether, Dimethiconol, Dipentaerythrityl Hexacaprylate/Hexacaprate, Domiphen Bromide, Ectoin, Galactosyl Fructose, one or more Glycosaminoglycans, Hexyldecanol, Hexyldecyl Isostearate, Hexyldecyl Laurate, one or more Hydrogenated C₆₋₁₄ Olefin Polymers, Hydrogenated Didecene, Hydrogenated Polydecene, Hydrolyzed Collagen, Hydrolyzed Elastin, Hydrolyzed Oat Protein, Isoamyl Laurate, Isobutyl Stearate, Isodecyl Neopentanoate, Isodecyl Octanoate, Isododecane, Lignoceryl Erucate, Magnesium Ascorbyl Phosphate, Mannan, Myristyl Myristate, Octocrylene, Oleyl Betaine, Pentaerythrityl Tetracaprylate/Tetracaprate, Pentaerythrityl Tetraisostearate, Phytosterol, Phytosteryl Hydroxystearate, Phytosteryl Isostearate, Polyamide-7, Polydecene, Polydiethysiloxane, Polyisobutene, Potassium Lauroyl Hydrolyzed Soy Protein, Potassium Soyate, Propanediol, Pyridoxine Dioctenoate, Pyridoxine Dipalmitate, Retinyl Palmitate, Sea Cucumber Chondroitin Sulfate, Sericin, Shark Cartilage Chondroitin Sulfate, Stearamide AMP, Sucrose Cocoate, Sucrose Polybehenate, Sucrose Polysoyate, Tricaprin, Tridecyl Neopentanoate, Tridecyl Salicylate, Tridecyl Stearate, Tridecyl Trimellitate, Triheptanoin, Triisostearin, Trioctanoin, Tris (PPG-3 Benzyl Ether) Citrate, Triundecanoin, Ubiquinone, and combinations thereof.

22. The composition of any of claims 1-21, further comprising:
(e) an active ingredient suitable for use in the treatment of alopecia, dermatitis, basal cell carcinoma, acne, actinic keratosis, rosacea, or psoriasis, or is suitable for use as an anti-fungal agent, an anti-bacterial agent, or a local anesthetic.

23. The composition of claim 22, wherein the active ingredient is suitable for use in the treatment of alopecia, Allergic contact dermatitis, Seborrheic dermatitis, Irritant contact dermatitis, Atopic dermatitis, Neurodermatitis, Perioral dermatitis, Stasis dermatitis, Diaper dermatitis, Lichen simplex chronicus, Dyshidrotic dermatitis, Nummular dermatitis, Autosensitization dermatitis, Spongiotic dermatitis, Lichenoid dermatitis, basal cell carcinoma, blackheads, whiteheads, pustules, Acne Vulgaris, Acne Rosacea, Cystic Acne, Nodular Acne, Acne Inversa, Hormonal Acne, Acne conglobata, Acne fulminans, Gram Negative folliculitis, Nodulocystic acne, Pyoderma Faciale, Acne Cosmetica, Pomade Acne, Excoriated Acne, Acne Agminata, Acne excoriee, Hyperkeratotic actinic keratosis, Pigmented actinic keratosis, Lichenoid actinic keratosis, Atrophic actinic keratosis, Vascular Rosacea, Acne Rosacea, Pre Rosacea, Papulopustular Rosacea, Late Rosacea, Erythematotelangiectatic Rosacea, Ocular Rosacea, Phymatous Rosacea, Psoriasis vulgaris (Plague Psoriasis), Guttate Psoriasis, Inverse Psoriasis, Pustular Psoriasis, Erythrodermic Psoriasis, Nail Poriasis, Psoriasis of the scalp, Psoriatic Arthritis, Seborrheic Psoriasis, Palmoplantar Psoriasis or skin fold Psoriasis; or is suitable for use as an anti-fungal agent, an anti-bacterial agent, or a local anesthetic.

24. The composition of claim 22 or 23, wherein the active ingredient is Minoxidil, Ketoconazole, Pramoxine Hydrochloride, Lidocaine, a Corticosteroid, a topical antibiotic, Retinoic Acid, or Retinol.

25. The composition of claim 24, wherein the active ingredient is Minoxidil, Ketoconazole, Pramoxine Hydrochloride, Lidocaine, Augmented Betamethasone dipropionate, Clobetasol 17-Propionate, Diflorasone diacetate, Halobetasol propionate, Amcinonide, Betamethasone dipropionate, Betamethasone valerate, Desoximetasone, Diflucortolone valerate, Fluocinonlone acetonide, Fluocinonide, Fluticasone Propionate, Halcinonide, Mometasone furoate, Triamcinolone acetonide, Betamethasone benzoate, Betamethasone dipropionate, Betamethasone valerate, Clocortolone pivalate, Clobetasone 17-Butyrate, Desoximetasone, Fluocinolone acetonide, Flurandrenolide, Fluticasone propionate, Hydrocortisone butyrate, Hydrocortisone valerate, Mometasone furoate, Triamcinolone acetonide, Alclometasone dipropionate, Desonide, Dexamethasone, Dexamethasone sodium Phosphate, Fluocinolone acetonide, Hydrocortisone, Hydrocortisone acetate, Prednicarbate, Clindamycin, Erythromycin, Tetracycline, Metronidazole, Bacitracin, Bacitracin zinc, neomycin, polymyxin B sulfate, Gentamicin, Mupirocin, Retapamulin, Retinoic Acid, or Retinol.

26. The composition of any of claims 22-25, wherein:
the foam booster is selected from the group consisting of Cocoamidopropyl Betaine, Cocamidopropylamine Oxide, Sodium Lauroyl Sarcosinate, Cetearyl Alcohol, Lauramine Oxide, Laureth-2, Laureth-4, Microcrystalline Cellulose, PEG-150 Distearate, PVP, and combinations thereof;
the foaming agent is selected from the group consisting of Polysorbate 20, Polysorbate 40, Polysorbate 60, Polysorbate 80, Poloxamer 124, Poloxamer 188, Poloxamer 237, Poloxamer 338, Poloxamer 407, Brij L9(PEG 9 Lauryl ether), Brij L23(PEG 23 Lauryl ether), Sodium lauryl Sulphate, Sodium Laureth Sulphate, Disodium Cocoamphodiacetate, Disodium Lauryl Sulfosuccinate, Myristic Acid, Sodium Cocoyl Sarcosinate, Sodium Laureth-2 Sulfate, Sodium Laureth-3 Sulfate, Sodium Lauryl Sulfoacetate, Sucrose Stearate, Polyoxyl 40 Hydrogenated castor oil, Polyoxyl 60 Hydrogenated castor oil, and combinations thereof;
the solubilizer is selected from the group consisting of Water, Ethanol, Diethylene Glycol monomethyl ether, Glycerine, Propylene Glycol, Isopropyl alcohol, Polyethylene Glycols, Benzyl alcohol, Dimethyl Sulfoxide, PEG 40 Castor oil, PEG 40 Hydrogenated Castor Oil, PEG 60 Hydrogenated Castor Oil, Sorbitan Stearate, Oleyl Alcohol, and combinations thereof; and
the emollient, if present, is selected from the group consisting of Dimethicone, Cyclomethicone, Arachis Hypogaea (Peanut) Oil, Canola Oil, Carthamus Tinctorius (Safflower) Seed Oil, Cocos Nucifera (Coconut) Oil, Citrus Aurantium Amara (Bitter Orange) Peel Oil, Citrus Medica Limonum (Lemon) Peel Oil, Citrus Aurantifolia (Lime) Oil, Elaeis Guineensis (Palm) Oil, Glycine Soja (Soybean) Oil, Mentha Piperita (Peppermint) Oil, Olea Europaea (Olive) Oil, PEG-60 Hydrogenated Castor Oil, Papaver Somniferum (Poppy) Seed Oil, Ricinus Communis (Castor) Seed Oil, Sesamum Indicum (Sesame) Seed Oil, Zea Mays (Corn) Oil, Butyrospermum Parkii (Shea) Butter, Theobroma Cacao (Cocoa) Seed Butter, PEG-75 Lanolin, PEG-2 Stearate, PEG-8 Laurate, PPG-15 Stearyl Ether, PPG-20 MethylGlucose Ether Distearate, Lanolin, Lanolin Alcohol, Acetylated Lanolin Alcohol, Lecithin, Caprylic/Capric Triglyceride, Caprylic/Capric/Stearic Triglyceride, Hydrogenated Palm Glycerides, Glycol Stearate, Butylene Glycol, Hexylene Glycol, Propylene Glycol, Cetearyl Alcohol, Cetyl Alcohol, Myristyl Alcohol, Oleyl Alcohol, Stearyl Alcohol, Glyceryl Caprylate, Glyceryl Laurate, Glyceryl Isostearate, Glyceryl Oleate, Glyceryl Stearate, Glyceryl Distearate, Polyglyceryl-3 Oleate, Polyglyceryl-10 Oleate, Isostearic Acid, Lactic Acid, Sodium Lactate, Copernicia Cerifera (Carnauba) Wax, Cera Alba (Beeswax), Euphorbia Cerifera (Candelilla) Wax, Polyquaternium-7, Choleth-24, Methyl Gluceth-10, Methyl Gluceth-20, Diisopropyl Adipate, Methyl Laurate, Cetearyl Ethylhexanoate, Diethyl Sebacate, Ethylhexyl Hydroxystearate, Xanthan Gum, Isopropyl Myristate, Isopropyl Palmitate, Cetyl Palmitate, Octyldodecanol, Acacia, Butyl Stearate, Cholesterol, Cysteine HCl, Lauryl Lactate, Myristyl Lactate, Paraffinum Liquidum (Mineral) Oil, Petrolatum, Polybutene, PVP, Sorbitan Isostearate, Squalane, Tocopherol, Tricaprylin, Urea, Zea Mays (Corn) Starch, Hyaluronic Acid, Sodium Hyaluronate, and combinations thereof.

27. The composition of any of claims 1-26, further comprising one or more of the following:
(f) about 0.02% to about 2.5% (w/w) preservative;
(g) about 1% to about 25% (w/w) penetration enhancer;
(h) a pH-modifying agent.

28. The composition of claim 27, wherein the preservative is selected from the group consisting of Methyl Paraben, Ethyl Paraben, Propyl Paraben, Butyl Paraben, Sodium Methyl Paraben, Sodium Ethyl Paraben, Sodium Propyl Paraben, Phenoxyethanol, Benzyl Alcohol, Chloroxylenol, Imidazolidinyl Urea, Diazolidinyl Urea, Benzoic Acid, Dehydroacetic acid, Sorbic acid, Methylchloroisothiazolinone, Methylisothiazolinone, Sodium Benzoate, Potassium Sorbate, Glyceryl Laurate, Butylated Hydroxy Anisole, Benzalkonium chloride, Benzethonium Chloride, Isobutylparaben, Sodium Butyl Paraben, Phenylpropanol, Dichloro-m-xylenol, Anisic acid, Polyaminopropyl Biguanide, Dimethyloxazolidine, 7-Ethylbicyclooxazolidine, Benzisothiazolonone, Methyldibromo Glutaronitrile, Iodopropynyl Butylcarbamate, Trisodium Ethylenediamine Disuccinate, Tris (hydroxymethyl) nitromethane, Triclosan, Caprylyl Glycol, Ethylhexylglycerin, Sodium Usnate, Sodium Levulinate, Sodium Anisate, Sodium Dehydroacetate, Sodium Salicylate, Sodium Hydroxymethyl Glycinate, Piroctone Olamine, Chlorphenesin, Hexamidine Diisethionate, Glyceryl Undecylenate, and combinations thereof.

29. The composition of claim 27, wherein the penetration enhancer is selected from the group consisting of one or more sulfoxides, one or more Azones, one or more pyrrolidones, one or more Alcohols, one or more Glycols, one or more Fatty acids, one or more Non-ionic surfactants, one or more Essential oils, Isopropyl palmitate, Isopropyl myristate, Glyceryl mono laurate, Oleic acid, 1,3-Butylene glycol, Lecithin, Dipropylene glycol, Glyceryl monooleate, Methyl laurate, Padimate O, Octyl salicylate, Urea, Sesquiterpene, Dimethyl formamide, and combinations thereof.

30. The composition of claim 29, wherein the penetration enhancer is selected from the group consisting of Dimethylsulphoxide (DMSO), Laurocapram, 2-pyrrolidone, N-methyl-2-pyrrolidone, Ethanol, Decanol, Lauryl alcohol, Propylene Glycol, Diethylene Glycol, tetraethylene glycol, lauric acid, myristic acid, capric acid, polyoxyethylene-2-oleyl ether, polyoxyethylene-2-stearyl ether, eucalyptus oil, chenopodium oil, ylang-ylang oil, L-menthol oil, Isopropyl palmitate, Isopropyl myristate, Glyceryl mono laurate, Oleic acid, 1,3-Butylene glycol, Lecithin, Dipropylene glycol, Glyceryl monooleate, Methyl laurate, Padimate O, Octyl salicylate, Urea, Sesquiterpene, Dimethyl formamide, and combinations thereof.

31. The composition of any of claims 1-4, consisting essentially of:
about 1.0% to about 2.0% (w/w) Cocamidopropyl Betaine;
about 1.0% to about 3.0% (w/w) Polysorbate 80;
about 1.0% to about 3.0% (w/w) Polyoxylpropylene-polyoxylethylene Block Copolymer;
about 5.0% to about 10.0% (w/w) Propylene Glycol;
about 6.0% to about 10.0% (w/w) Ethyl Alcohol;
about 0.5% to about 1.5% (w/w) PEG-75 Lanolin;
about 1.0% to about 2.0% (w/w) Magnesium Ascorbyl Phosphate;
about 2.0% to about 4.0% (w/w) Arbutin;
and balance water.

32. The composition of any of claims 1-4, consisting essentially of:
about 1.0% to about 4.0% (w/w) Sodium Lauroyl Sarcosinate;
about 3.0% to about 5.0% (w/w) Polyoxylpropylene-polyoxylethylene Copolymer;
about 25.0% to about 35.0% (w/w) Propylene Glycol;
about 2.0% to about 8.0% (w/w) Ethyl Alcohol;
about 0.01% to about 0.1% (w/w) Citric Acid;
about 0.1% to about 1.0% (w/w) Lactic Acid;
about 2.0% to about 5.0% (w/w) Minoxidil;
about 1.0% to about 3.0% (w/w) Diethylene Glycol Monoethyl Ether;
about 1.0% to about 2.0% (w/w) Polyoxy lauryl Ether;
about 0.01% to about 0.1% (w/w) Benzyl Alcohol;
and balance water.

33. The composition of any of claims 1-4, consisting essentially of:
about 1.0% to about 2.0% (w/w) Cocamidopropylamine Oxide;
about 0.1% to about 1.0% (w/w) PEG 40 Hydrogenated Castor Oil;
about 6.0% to about 10.0% (w/w) Propylene Glycol;
about 5.0% to about 10.0% (w/w) Isopropyl Alcohol;
about 1.0% to about 2.0% (w/w) PEG-6 Caprylic/Capric Glyceride;
about 0.05% to about 0.10% (w/w) Clobetasol Propionate;
and balance water.

34. The composition of claim 14, wherein:
(a) the foam booster consists essentially of about 3% to about 6% (w/w) cocoamidopropyl betaine;
(b) the foaming agent consists essentially of about 0.2% to about 1.5% (w/w) polysorbate 80 and about 1.5% to about 3.5% (w/w) polyoxyethylene-polyoxypropylene copolymer;
(c) the solubilizer consists essentially of about 5% to about 10% (w/w) ethyl alcohol (95%) and about 6% to about 10% (w/w) propylene glycol; and
(d) the emollient consists essentially of about 1% to about 2% (w/w) PEG 75 lanolin;
and the composition comprises balance water;
wherein the composition optionally further comprises one or more of the following:
(e) an active ingredient suitable for use in the treatment of alopecia, dermatitis, basal cell carcinoma, acne, actinic keratosis, rosacea, or psoriasis, or is suitable for use as an anti-fungal agent, an anti-bacterial agent, or a local anesthetic;
(f) about 0.02% to about 2.5% (w/w) preservative;
(g) about 1% to about 25% (w/w) penetration enhancer;
(h) a pH-modifying agent.

35. The composition of claim 14, wherein:
(a) the foam booster consists essentially of about 1% to about 2% (w/w) cocoamidopropylamine oxide;
(b) the foaming agent consists essentially of about 0.2% to about 1.5% (w/w) polysorbate 80 and about 0.2% to about 0.4% (w/w) polyoxyethylene-polyoxypropylene copolymer;
(c) the solubilizer consists essentially of about 5% to about 10% (w/w) ethyl alcohol (95%) and about 10% to about 20% (w/w) propylene glycol; and
(d) the emollient consists essentially of about 1% to about 2% (w/w) PEG 6 caprylic/capric glyceride;
and the composition comprises balance water;
wherein the composition optionally further comprises one or more of the following:
(e) an active ingredient suitable for use in the treatment of alopecia, dermatitis, basal cell carcinoma, acne, actinic keratosis, rosacea, or psoriasis, or is suitable for use as an anti-fungal agent, an anti-bacterial agent, or a local anesthetic;
(f) about 0.02% to about 2.5% (w/w) preservative;
(g) about 1% to about 25% (w/w) penetration enhancer;
(h) a pH-modifying agent.

36. The composition of claim 14, wherein:
(a) the foam booster consists essentially of about 3% to about 7% (w/w) sodium lauroyl sarcosinate;
(b) the foaming agent consists essentially of about 1% to about 2% (w/w) PEG 40 hydrogenated castor oil and about 1.0% to about 2.5% (w/w) polyoxy lauryl ether;
(c) the solubilizer consists essentially of about 5% to about 10% (w/w) ethyl alcohol (95%) and about 10% to about 20% (w/w) propylene glycol; and
(d) the emollient consists essentially of about 0.5% to about 1% (w/w) sodium salt of pyrrolidone carbonic acid;
and the composition comprises balance water;
wherein the composition optionally further comprises one or more of the following:
(e) an active ingredient suitable for use in the treatment of alopecia, dermatitis, basal cell carcinoma, acne, actinic keratosis, rosacea, or psoriasis, or is suitable for use as an anti-fungal agent, an anti-bacterial agent, or a local anesthetic;
(f) about 0.02% to about 2.5% (w/w) preservative;
(g) about 1% to about 25% (w/w) penetration enhancer;
(h) a pH-modifying agent.

37. A non-aerosol pump for delivering a foam, and contained within the pump a liquid composition suitable for topical administration and for use with a non-aerosol pump, wherein actuation of the pump produces a foam, wherein the composition is the composition of any of claims 1-36.

38. A foam composition having a density of about 0.076 to about 0.102 g/mL, which foam composition is produced by a method comprising passing a liquid composition through a non-aerosol pump, wherein the liquid composition is the liquid composition of any of claims 1-36.

39. A foam composition characterized in that the time for the foam produced to dissipate into a liquid is about 45 minutes or longer for a volume of about 40 mL of foam, which foam composition is produced by a method comprising passing a liquid composition through a non-aerosol pump, wherein the liquid composition is the liquid composition of any of claims 1-36.

40. A method of applying to the dermis a foam composition having a density of about 0.076 to about 0.102 g/mL, which foam composition is produced by a method comprising passing a liquid composition through a non-aerosol pump, wherein the liquid composition is the liquid composition of any of claims 1-36.

41. A method of applying to the dermis a foam composition characterized in that the time for the foam produced to dissipate into a liquid is about 45 minutes or longer for a volume of about 40 mL of foam, which foam composition is produced by a method comprising passing a liquid composition through a non-aerosol pump, wherein the liquid composition is the liquid composition of any of claims 1-36.
